(19) United States
(12) Patent Application Publication
(10) Pub. No.: US 2016/0031925 A1
(43) Pub. Date: Feb. 4, 2016

(54) AZITHROMYCIN ANTIMICROBIAL DERIVATIVES WITH NON-ANTIBIOTIC PHARMACEUTICAL EFFECT

(71) Applicant: PROBIOTIC PHARMACEUTICALS APS, KGS. Lyngby (DK)

(72) Inventors: Friõrik GARDARSSON, Reykjavik (IS); Susanne GULDBERG, Lyngby (DK); Magnús GARDARSSON, Lyngby (DK)

(21) Appl. No.: 14/783,245

(22) PCT Filed: Apr. 10, 2014
(86) PCT No.: PCT/DK2014/050092

§ 371 Date: Oct. 8, 2015

(30) Foreign Application Priority Data
Apr. 10, 2013 (IS) 050050

(51) Int. Cl. C07H 17/00; C07D 413/14

(52) U.S. Cl. CPC C07H 17/00; C07D 413/14

(57) ABSTRACT

The invention provides molecules, which are based on a modification of azithromycin, removing the antibiotic effect, while retaining other beneficial effects, such as, but not limited to immunomodulatory effects. The compounds of the invention can be described by compounds of Formula (I) as further defined herein.

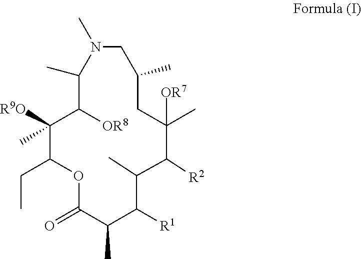

### FIELD OF INVENTION

The present invention concerns new molecules, which are based on a modification of azithromycin, removing the antibiotic effect, while retaining other beneficial effects, such as, but not limited to immunomodulatory effects.

### TECHNICAL BACKGROUND AND PRIOR ART

Azithromycin is an antibiotic drug whose activity stems from the presence of a 15 membered macrolide ring, to which the sugars, cladinose and desosamine are attached. Azithromycin is used to treat bacteriologic infections caused by Gram-positive bacteria and Haemophilus infections such as respiratory tract and soft-tissue infections. Thus, Azithromycin is primarily used to treat or prevent certain bacterial infections, most often those causing middle ear infections, strep throat, pneumonia, typhoid, gastroenteritis, bronchitis and sinusitis, Azithromycin is also found effective against certain sexually transmitted infections, such as nongonococcal urethritis, chlamydia, and cervicitis.

WO2012131396, WO2006087644, WO9900124, EP283055 describe various derivatives of azithromycin and relates to the identification of new and/or robust compounds of azilthromycin having antibacterial activity. WO2007093840, US20060183696, WO2006046123, WO2003070254 describe various conjugates having an azilthromycin moiety and concerns the treatment of inflammatory diseases.

Chronic obstructive pulmonary disease (COPD) is described by the progressive development of airflow limitation that is not fully reversible. Most patients with COPD have three pathological conditions: bronchitis, emphysema and mucus plugging. This disease is characterized by a slowly progressive and irreversible decrease in forced expiratory volume in the first second of expiration (FEV1), with relative preservation of forced vital capacity (FVC) (Barnes, N. Engl. J. Med. (2000), 343(4): 269-280). In both asthma and COPD there is significant, but distinct, remodeling of airways. Most of the airflow obstruction is due to two major components, alveolar destruction (emphysema) and small airways obstruction (chronic obstructive bronchitis). COPD is mainly characterized by profound mucus cell hyperplasia. The group of inflammatory diseases includes amongst other chronic obstructive pulmonary disease, adult respiratory distress syndrome, some types of immune-complex alveolitis, cystic fibrosis, bronchitis, bronchiectasis, and emphysema, etc. In these conditions neutrophils are thought to play a crucial role in the development of tissue injury which, when persistent, can lead to the irreversible destruction of the normal tissue architecture with consequent organ dysfunction. Tissue damage is primarily caused by the activation of neutrophils followed by their release of proteinases and increased production of oxygen species.

Apart from azithromycins antibiotic properties, azithromycin has also an established beneficial effect on the respiratory function and survival among patients with diffuse panbronchiolitis (1, 2) and cycstic fibrosis and other chronic lung diseases, independently of the antibiotic effect and frequency of infections (3, 4, 5). It has been suggested that azithromycin may increase the transepithelial electrical resistance of human airway epithelia by changing the processing of tight junction proteins. In particular, azilthromycin may have a positive impact on the tetraspan transmembrane proteins, such as claudin-1, claudin-4, and occludin, (6). A corresponding beneficial effect observed for azilthromycin was not observed for neither penicillin nor erythromycin. Too much use of antibiotics in human and animals is a serious concern as this is believed to be a contributing factor to increased antibiotic resistance and multi-drug resistant bacteria. Therefore, it is not generally advisable to administer antibiotic compounds for other potential medical effects than bacterial infections. If however, the antibacterial effect of azilthromycin can be repressed, quelled or diminished while maintaining the other beneficial effects, this could be of high medical importance.

### SUMMARY OF INVENTION

The present inventors have, based on other beneficial effects of azithromycin, developed new compounds, which have been modified to reduce or eliminate the antibiotic effect that azithromycin exhibits, while retaining other beneficial effects, such as, but not limited to immunomodulatory effects, increased processing of tight junction proteins and improved transepithelial. The present invention provides novel compounds with this effect, and thereby creates the possibility to introduce a new drug, which could enable a non-antibiotic novel treatment of cystic fibrosis, COPD, bronchiolitis, and possibly other respiratory related diseases, which could greatly reduce the unnecessary use of antibiotics and related problems with bacteria forming resistance towards these antibiotics.

In a first aspect the invention provides new compounds defined by Formula (I), as further defined herein. Non-limiting examples of suitable compounds of the invention are set forth in the Examples section.

In another aspect the invention provides pharmaceutical compositions of the compounds of the invention, described further herein.

The compounds of the invention can be synthesised as described in detail in the accompanying examples.

As mentioned above, the compounds of the invention can be generally described by Formula (I)

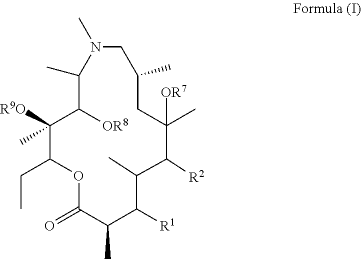

wherein
R¹ is OH, CH₃, OCH₃, a C₂-C₄ straight or branched alkyl group, or the group R³, which is bounded to Formula (I) via a covalent bonding to oxygen, where R⁵ is H, OH or CH₃, Formula (I)

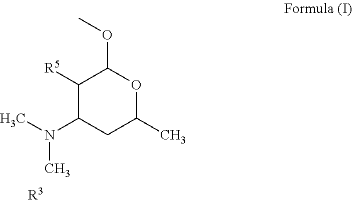

R² is OH, CH₃, OCH₃, a C₂-C₄ straight or branched alkyl group, or the group R⁴, which is bounded to Formula (I) via a covalent bonding to oxygen, where R⁶ is H, OH or CH₃,

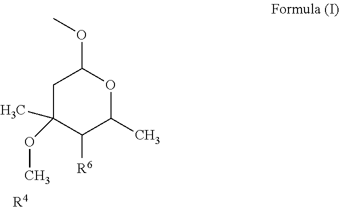

R⁷ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
R⁸ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
R⁹ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine, halogen is Cl, Br, or I,
or a pharmaceutically derivative thereof, tautomers and stereoisomers thereof, or a pharmaceutically acceptable salt thereof, and
with the provisio that
R⁵ and R⁶ cannot both be OH.

In a specific embodiment, the present invention relates to compounds of Formula (II)

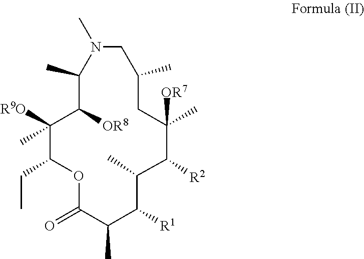

wherein
R¹ is OH, CH₃, OCH₃, a C₂-C₄ straight or branched alkyl group, or the group R³, which is bounded to Formula (I) via a covalent bonding to oxygen,

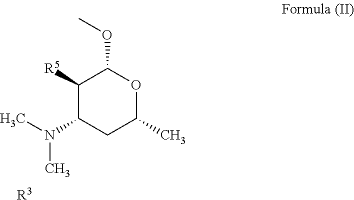

R² is OH, CH₃, OCH₃, a C₂-C₄ straight or branched alkyl group, or

the group R⁴, which is bounded to Formula (I) via a covalent bonding to oxygen,

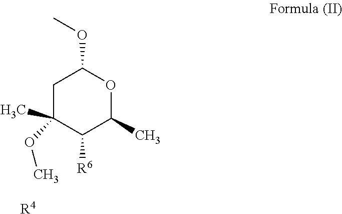

R⁵, R⁶, R⁷, R⁸, and R⁹ has the same meanings as given above,

or a pharmaceutically derivative thereof, tautomers and stereoisomers thereof, or a pharmaceutically acceptable salt thereof,
with the provisio that
R⁵ and R⁶ cannot both be OH.

In one specific embodiment of the present invention, compounds of Formula (I) and of Formula (II), wherein R¹ is the group R³, where R⁵ is H, OH or CH₃, and R² is OH, CH₃, or OCH₃, is preferred.

In another embodiment of the present invention, compounds of Formula (I) and of Formula (II), wherein R¹ is OH, CH₃, OCH₃, and R² is the group R⁴, with R⁶ being H, OH or CH₃, is preferred.

In yet another embodiment of the present invention, compounds of Formula (I) and of Formula (II), wherein
i) R¹ is the group R³, with R⁵ being CH₃, and R² is the group R⁴, with R⁶ being OH;
ii) R¹ is the group R³, with R⁵ being OH and R² is the group R⁴, with R⁶ being CH₃;
iii) R¹ is the group R³, with R⁵ being CH₃ and R² is the group R⁴, with R⁶ being CH₃;
iv) R¹ is the group R³, with R⁵ being OH and R² is the group R⁴, with R⁶ being H;
v) R¹ is the group R³, with R⁵ being H and R² is the group R⁴, with R⁶ being OH;
vi) R¹ is the group R³, with R⁵ being H and R² is the group R⁴, with R⁶ being H;
vii) R¹ is the group R³, with R⁵ being CH₃ and R² is the group R⁴, with R⁶ being H;
viii) R¹ is the group R³, with R⁵ being H and R² is the group R⁴, with R⁶ being CH₃;
ix) R¹ is OH and R² is OH;
x) R¹ is CH₃ and R² is CH₃;
xi) R¹ is OCH₃ and R² is OCH₃;
xii) R¹ is OH and R² is the group R⁴, with R⁶ being CH₃;
xiii) R¹ is CH₃ and R² is the group R⁴, with R⁶ being CH₃;
xiv) R¹ is the group R³, with R⁵ being OH and R² is CH₃;
xv) R¹ is the group R³, with R⁵ being any methyl- or ethyl ester and R² is CH₃;
xvi) R¹ is the group R³, with R⁵ being CH₃ and R² being any methyl- or ethyl ester,
   are particularly preferred.

Further, in one specific embodiment of the present invention, relating to the compounds of Formula (I) and compounds of Formula (II) as such,
R¹ and R² cannot both be OH,
R¹ cannot be OH when R⁶ is OH,
R² cannot be OH when R⁵ is OH, or
R⁵ cannot be H when R⁶ is OH.

Preferred embodiments of the invention are shown in the chemical Formulae below, as compounds PP001 to PP008.

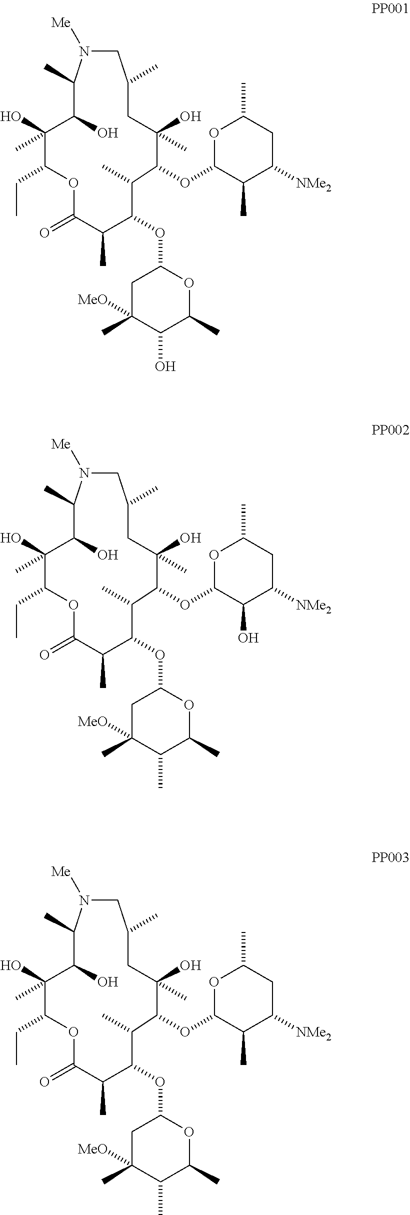

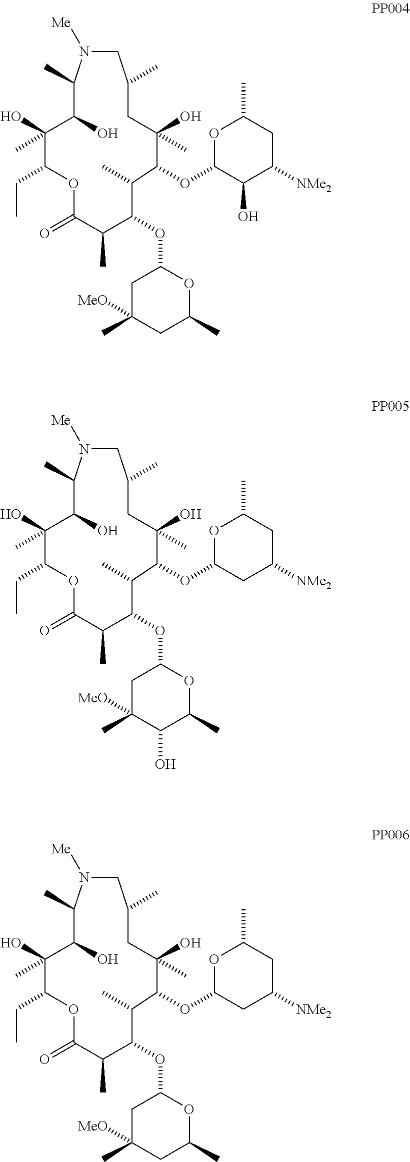

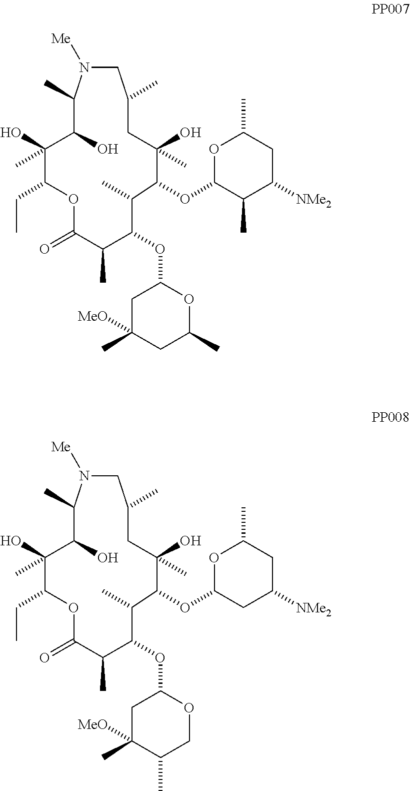

### DETAILED DESCRIPTION

The compounds of the present invention may be in the form of and/or may be administered as a pharmaceutically acceptable salt. For a review on suitable salts see Berge et ah, J. Pharm. ScL, 1977, 66, 1-19. Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as hydrochloric acid may be added to an aqueous suspension of a compound of Formula (I) and the resulting mixture evaporated to dryness (lyophilised) to obtain the acid addition salt as a solid. Suitable addition salts are formed from inorganic or organic acids which form non-toxic salts and examples are, but not limited to, hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulfonates (e.g. methanesulfonate, ethanesulfonate, benzenesulfonate or p-toluenesulfonate) and isothionate. Representative examples include, but are not limited to, trifluoroacetate and formate salts, for example the bis- or tris-trifluoroacetate salts and the mono or diformate salts, in particular the bis- or tris-trifluoroacetate salt and the monoformate salt.

The compounds of Formula (I) may be in crystalline or amorphous form. Furthermore, some of the crystalline forms of the compounds of Formula (I) may exist as polymorphs, which are included in the present invention. Organic molecules can form crystals that incorporate water into the crystalline structure without modification of the organic molecule. An organic molecule can exist in different crystalline forms, each different crystalline forms may contain the same number of water molecules pr organic molecule or a different number of water molecules pr organic molecule.

In addition, some of the compounds may form solvates with water (i.e. hydrates) or common organic solvents, and such solvates are also intended to be encompassed within the scope of this invention. The compounds, including their salts, can also be obtained in the form of their hydrates, or include other solvents used for their crystallization.

The compounds of Formula (I) and Formula (II) may be in the form of a prodrug. The term “prodrug” as used herein means a compound which is converted within the body, e.g. by hydrolysis in the blood, into its active form that has medical effects. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of one or more of alcohol, sulfhydryl and amine functional groups of the compounds of structure (I). Further, in the case of a carboxylic acid (—COOH) group, esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

The term alkyl as used herein as a group or a part of a group refers to a straight or branched hydrocarbon chain containing the specified number of carbon atoms. Examples of such group include but are not limited to methyl, ethyl, propyl, isopropyl, n-butyl, isobutyl, tert-butyl, pentyl, 3-methyl-butyl, hexyl and 2,3-dimethylbutyl and like.

The term “alkenyl”, unless otherwise indicated, may be interpreted similarly to the term “alkyl”. Alkenyl groups contain at least 1 double bond. Suitable alkenyl groups include ethenyl, propenyl, 1-butenyl, and 2-butenyl.

The term “alkynyl”, unless otherwise indicated, may be interpreted similarly to the term “alkyl”. Alkenyl groups contain at least 1 triple bond.

The term “saturated or unsaturated C₅- or C₆-cycloalkyl”, unless otherwise indicated, denotes cyclic carbon rings comprising 5 or 6 carbon atoms, wherein either a single or double bond between the mutually adjacent carbon atoms exist.

Suitable saturated or unsaturated C₅- or C₆-cycloalkyl groups include cyclopentane, cyclohexane, cyclopentene, cyclohexene, cyclopenta-diene, cyclohhexadiene, and phenyl.

The term “5- or 6-membered heterocyclyl”, unless otherwise indicated, denotes a heterocyclic compound, such as a carbocyclyl group, phenyl group, or aryl residue, having atoms of at least two different elements as members of its ring. Suitable ring atoms in heterocyclic compound may be C, N, S, or O.

Heterocyclic compounds according to the present invention may contain 3, 4, 5, 6, 7, 8 or even more rings atoms, preferably 5 or 6 ring atoms.

The term “halogen” comprises fluorine (F), chlorine (Cl), bromine (Br) and iodine (I), more typically Cl or Br.

All possible tautomers of the claimed compounds are included in the present invention. Tautomers are isomers of organic compounds that readily interconvert by a chemical reaction called tautomerization. This reaction commonly results in the formal migration of a hydrogen atom or proton, accompanied by a switch of a single bond and adjacent double bond.

The compounds of the present invention have several asymmetric centers. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all steric forms of the compounds of the invention. The present invention includes all stereoisomers of compounds of Formula (I).

The independent syntheses of the stereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration. If desired, racemic mixtures of the compounds may be separated so that the individual enantiomers or diastereomers are isolated. The separation can be carried out by methods well known in the art, such as the coupling of a racemic mixture of compounds, followed by separation of the individual stereisomers by standard methods, such as fractional crystallization or chromatography. The coupling reaction is often the formation of salts using an enantiomerically pure acid or base.

The derivatives may then be converted to the pure stereomers by cleavage of the added chiral residue. The racemic mixture of the compounds can also be separated directly by chromatographic methods using chiral stationary phases, which methods are well known in the art.

Alternatively, any stereomers of a compound may be obtained by stereoselective synthesis using optically pure starting materials or reagents of known configuration by methods well known in the art. “Treating” or “treatment” of a state, disorder or condition includes:

(i) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition,
(ii) inhibiting the state, disorder or condition, i.e., arresting or reducing the development of the disease or at least one clinical or subclinical symptom thereof, or
(iii) relieving the disease, i.e., causing regression of the state, disorder or condition or at least one of its clinical or subclinical symptoms.
The benefit to a subject to be treated is either statistically significant or at least perceptible to the patient or to the physician.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “subject” refers to an animal, preferably a mammal, most preferably a human, who has been the object of treatment, observation or experiment. Treatment of animals, such as mice, rats, dogs, cats, cows, sheep and pigs, is, however, also within the scope of the present invention.

In another aspect the present invention relates to pharmaceutical compositions containing an effective dose of compounds of the present invention as well as pharmaceutically acceptable excipient, such as a carrier or diluent. The pharmaceutically acceptable carrier is suitably selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Pharmaceutical carriers according to the invention can be sterile liquids, such as but not limited to water, saline solutions, aqueous dextrose solutions, aqueous glycerol solutions; and/or oils, including petroleum, animal, vegetable or synthetic origin, such as soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

In yet a certain embodiment, the present invention relates to compounds of Formula (I) and compounds of Formula (II), pharmaceutical compositions thereof, or methods, for treatment of disorders of for use in treatment of asthma, COPD, diffuse panbronchiolitis, adult respiratory distress syndrome, inflammatory bowel disease, Crohn's disease, chronic bronchitis, and cystic fibrosis.

It will be appreciated that pharmaceutical compositions for use in accordance with the present invention may be in the form of oral, parenternal, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. It is to be understood that not all of the compounds need to be administered by the same route.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Syn thesis, John Wiley & Sons, 1991, fully incorporated herein by reference. The protecting groups may be removed at a convenient subsequent stage using methods known from the art.

Test for the antimicrobial activity of the novel compounds may be performed according to the standards of Clinical and Laboratory Standards Institute, Performance Standards for Antimicrobial Disk Susceptibility Tests; approved Standard; M2-A), vol. 26 NO. 1 9^{th} ed.

In one embodiment of the present invention, compounds of Formula (I) and Formula (II) show a 25% reduction in the response compared to an antibiotic reference, when testing for the antimicrobial activity of the novel compounds according to the standards of Clinical and Laboratory Standards Institute, Performance Standards for Antimicrobial Disk Susceptibility Tests; approved Standard; M2-A), vol. 26 NO. 1 9^{th} ed. Other relevant antibiotic assays may be used as well. The antibiotic reference may be selected between gentamycin, ampicillin, chloramphenicol, penicillin, or any other suitable antibiotic. In yet a preferred embodiment of the present invention, compounds of Formula (I) and Formula (II) show a 30%, 50%, 755, 85%, 90%, 95% or even higher reduction in the response compared to an antibiotic reference.

In one embodiment of the present invention, compounds of Formula (I) and Formula (II) are tested for their properties regarding maintainance of the non-antibiotic properties of azilthromycin. In yet another embodiment, compounds of Formula (I) and Formula (II) maintains at least 50%, 60%, 70%, 75%, 80%, 90, 95% of the non-antibiotic properties of azilthromycin are maintained by the novel compounds of Formula (I) and Formula (II), preferably more than 75%, even preferably more than 90%. Alternatively, the testing of the maintainance of the non-antibiotic properties of azilthromycin may result in a positive/negative evaluation or indication.

Suitable assay for testing of the non-antibiotic properties of azilthromycin would be, but not limited to, measurement on, e.g. human lung cells, for processing of tight junction proteins claudin-1, claudin-4, occludin and JAM-A and how they affect the cells transepithelial electrical resistance (TER) assays as a measure for strengthened intercellular epithelial coherence, or immunomodulating assays, or the methods as applied in references 1, 2, 3, 4, 5 or 6, which hereby is incorporated by reference. Protocols for any of these assays are well-known to the skilled person.

### EXAMPLES

### Example 1

### Synthesis of Compound PP001

PP001 is synthesized in 13 steps according to the description below.

A. Synthesis of the Benzoate 7

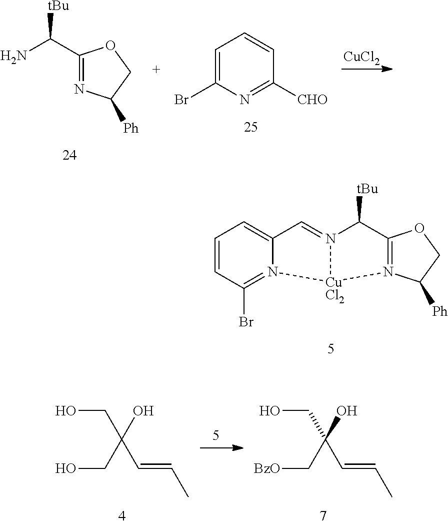

Phenyloxazolineamine 24 (0.40 mmol) in CH₂Cl₂ (5 mL) was reacted with romopyridinecarboxaldehyde 25 (0.40 mmol) in the presence of MgSO₄ (1.99 mmol) at room temperature for 1 hour. Then, CuCl₂ (0.40 mmol) was added and stirred at that temperature for another hour. The mixture as filtered through celite (700 mg) and evaporated in vacuo to generate the catalyst 5. After dissolving the remaining residue in THF (20 mL), the triol 4 (2.00 mmol) in THF (6 mL), Et₃N (2.44 mmol) and benzoyl chloride (2.22 mmol) were added to the catalyst 5 at room temperature in sequence, and then the mixture was stirred at the same temperature for 30 minutes. Quenching the benzoylation with saturated aqueous NH₄Cl (10 mL), work-up with EtOAc (10 mL×3) and the final chromatographic separation (EtOAc/hexane=1/2) produced the monobenzoate 7.

B. Synthesis of the Hydroxyl Epoxide 9

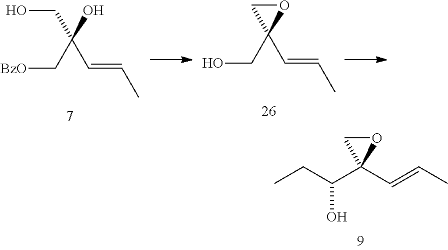

To the benzoate 7 (2.12 mmol) dissolved in CH₂Cl₂ (5 mL) were added MeSO₂Cl (2.58 mmol) and Et₃N (2.72 mmol) at −78° C., and then the mixture was stirred at that temperature for 15 minutes. After raising the reaction temperature to room temperature, DBU (2.57 mmol) in CH₂Cl₂ (1 mL) was injected to the mixture. The resulting solution was stirred for 6 hours at that temperature, and then quenched with saturated aqueous NH₄Cl (3 mL). Normal work-up with CH₂Cl₂ (3 mL×2) and the following column chromatography (Et2O/hexane=1/10) afforded the epoxy benzoate. The epoxy benzoate (1.65 mmol) was dissolved in MeOH (1.5 mL), and subsequently K₂CO₃ (0.25 mmol) was added. After stirring the mixture at room temperature for 2 hours, the reaction was quenched with saturated aqueous NH₄Cl (5 mL). Work-up with CH₂Cl₂ (2 mL×3) and chromatographic purification (EtOAc/hexane=1/5) furnished the epoxy alcohol 26. To 26 (1.47 mmol) in a mixture of DMSO (1 mL) and CH₂Cl₂ (3 mL) were added Et3N (11.76 mmol) and S₃Py (11.76 mmol) at 0° C., and the mixture was stirred for 1 hour at that temperature. Work-up was carried out by addition of H₂O (4 mL), extraction with Et₂O (3 mL×3), washing with 0.5 M HCl (2 mL) and brine (2 mL), drying over MgSO₄ (500 mg), filtration, and evaporation of all the volatile materials in vacuo to yield the crude epoxy aldehyde. Et₂Zn (1.0 M in hexane, 2.94 mmol) and the crude aldehyde in toluene (0.5 mL) were sequentially injected to the amino alcohol 8 (16 mg) in toluene (2 mL) at 0° C. After removal of the ice bath, the resulting solution was stirred at room temperature for 24 hours, and then quenched with 1 M HCl (2 mL). Normal work-up with Et₂O (4 mL×3) and the ensuing chromatographic separation (Et₂O/hexane=1/7) gave the epoxy alcohol 9 and its diastereomer.

C. Synthesis of the Epoxide 10

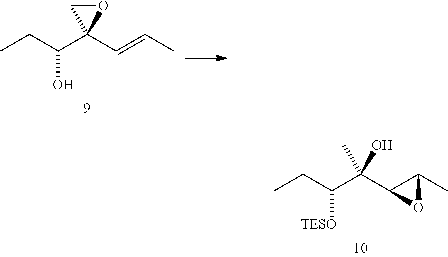

To 9 (2.11 mmol) in THF (2 mL) was added Vitride® (65 wt % in toluene, 2.53 mmol) diluted in THF (2 mL) at 0° C. and the mixture was stirred at that temperature for 8 hours. After quenching the reduction with 1 M H₂SO₄ (2 mL), usual work-up with Et₂O (3 mL×3), and the following column chromatography (Et₂O/hexane=1/3) provided the diol. Triethylsilyl chloride (2.21 mmol) and imidazole (2.80 mmol) were added to the diol (1.86 mmol) in DMF (2 mL) at room temperature in sequence and the resulting solution was stirred at that temperature for 8 hours. The silylation was quenched with H₂O (2 mL), work-up with Et₂O (3 mL×3) and the crude product was separated chromatographically (EtOAc/hexane=1/8) to render the TES ether. To the TES ether (1.75 mmol) in CH₂Cl₂ (4 mL) was added m-chloroperbenzoic acid (77% purity, 2.63 mmol) at −50° C. and the mixture was stirred at that temperature for 6 hours. After quenching the epoxidation with 1 M aqueous NaOH (2 mL), usual work-up with EtOAc (3 mL×3) and chromatographic purification (EtOAC/hexane=1/6) gave rise to the silyloxy epoxide 10.

D. Synthesis of the amine 2

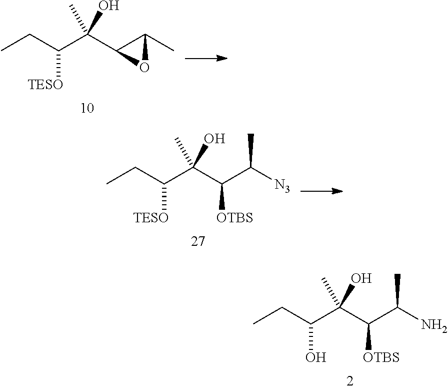

NaN₃ (3.64 mmol) and MgSO₄ (3.64 mmol) were added to 10 (1.82 mmol) in 2-methoxyethanol (5 mL) at room temperature, and the resulting mixture was heated at 11° C. for 6 hours. After cooling the mixture to room temperature, it was filtered through celite (500 mg) with EtOAc (10 mL). The filtrate was evaporated in vacuo and the residue was separated chromatographically (EtOAc/hexane=1/4) to impart the hydroxyl azide. To the hydroxyl azide (1.49 mmol) in DMF (4 mL) were added t-butyldimethylsilyl chloride (2.10 mmol) and imidazole (2.38 mmol) at room temperature for 5 hours. Quenching the silylation with H₂O (2 mL), work-up with Et₂O (4 mL×3) and column chromatography (Et₂O/hexane=1/30) supplied the TBS ether azide 27. Ph₃P (2.68 mmol) was added to 27 (1.34 mmol) in a 10:1 mixture of THF and H₂O (4.4 mL) at room temperature, and the solution was stirred at that temperature for 10 hours. After evaporation of the volatile materials in vacuo, the residue was purified chromatographically (Et₂O/hexane=1/10) to procure the silyl ether amine. Pyridinium fluoride in a mixture of THF (6 mL) and pyridine (60 μL) was injected to the silyl ether amine (1.17 mmol) in THF (2 mL) at 0° C., and the mixture was stirred at room temperature for 5 hours. After addition of saturated aqueous NaHCO₃ (2 mL), normal work-up with EtOAc (3 mL×3) and column chromatography (EtOAc/hexane=1/2) delivered the amine 2.

E. Synthesis of the triol 13

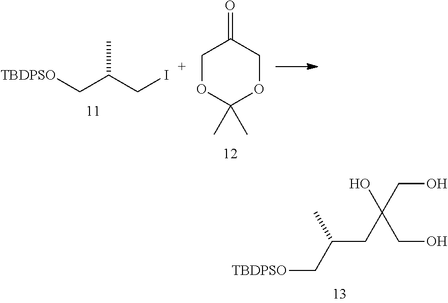

To a mixture of the iodide 11 (3.07 mmol) and the ketone 12 (3.07 mmol) in THF (16 mL) was added s-BuLi (1.4 M in cyclohexane, 5.53 mmol) dropwise at −98° C. The reaction solution was stirred at −98° C. for 2 hours and then quenched with saturated aqueous NH₄Cl (10 mL). Normal work-up with Et₂O (5 mL×3) and chromatographic separation (Et₂O/hexane=1/10) offered the adduct acetonide. Propane-1,3-dithiol (5.03 mmol) and BF₃.OEt₂ (0.12 mmol) were added to the adduct acetonide (1.93 mmol) in CH₂Cl₂ (5 mL) at 0° C., and then the mixture was stirred at 0° C. for 1 hour. Quenching the hydrolysis with saturated aqueous NaHCO₃ (3 mL), work-up with EtOAc (4 mL×3) and column chromatography (MeOH/CH₂Cl₂=1/15) afforded the triol 13.

F. Synthesis of the monobenzoate 14

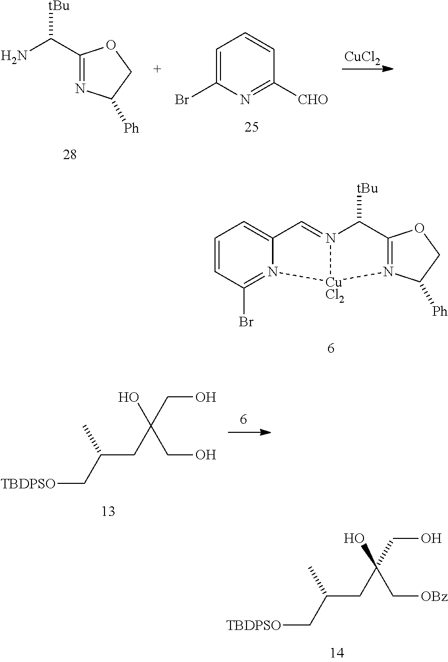

Phenyloxazolineamine 28 (0.20 mmol) in CH₂Cl₂ (3 mL) was reacted with bromopyridinecarboxaldehyde 25 (0.20 mmol) in the presence of MgSO₄ (0.99 mmol) at room temperature for 1 hour. Then, CuCl₂ (0.20 mmol) was added and stirred at that temperature for another hour. The mixture was filtered through celite (400 mg) and evaporated in vacuo to generate the catalyst 6. After dissolving the remaining residue in THF (10 mL) the triol 13 (1.00 mmol) in THF (3 mL), Et₃N (1.20 mmol) and benzoyl chloride (1.10 mmol) were added to the catalyst 6 at room temperature in sequence, and then the mixture was stirred at the same temperature for 30 minutes. Quenching the benzoylation with saturated aqueous NH₄Cl (5 mL), work-up with EtOAc (5 mL×3) and the following chromatographic separation (EtOAc/hexane=1/4) produced the monobenzoate 14 and its diastereomeric monobenzoate.

G. Synthesis of the Epoxy Alcohol 16

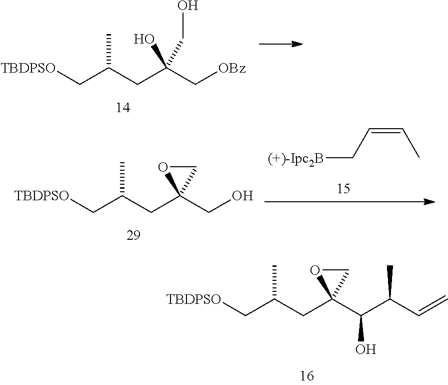

To the monobenzoate 14 (1.38 mmol) in CHBCl₂ (5 mL) were added MeSO₂Cl (1.67 mmol) and Et₃N (1.80 mmol) at −78° C., and then the mixture was stirred at that temperature for 30 minutes. After raising the reaction temperature to room temperature, DBU (1.66 mmol) in CH₂Cl₂ (1 mL) was injected to the mixture. The resulting solution was stirred for 6 hours at that temperature, and then quenched with saturated aqueous NH₄Cl (3 mL). Normal work-up with CH₂Cl₂ (3 mL×3) and the following column chromatography (Et₂O/hexane=1/15) yielded the epoxy benzoate. The epoxy benzoate (1.08 mmol) was dissolved in MeOH (3 mL), and subsequent K₂CO₃ (0.16 mmol) was added. After stirring the mixture at room temperature for 2 hours, the reaction was quenched with saturated aqueous NH₄Cl (2 mL). Work-up with CH₂Cl₂ (2 mL×3) and chromatographic purification (EtOAc/hexane=1/9) gave the epoxy alcohol 29. To 29 (0.97 mmol) in a mixture of DMSO (1 mL) and CH₂Cl₂ (3 mL) were added Et₃N (1.1 mL, 7.79 mmol) and SO₃Py (7.78 mmol) at 0° C., and the mixture was stirred for 1 hour at that temperature. Work-up was carried out by addition of H₂O (4 mL), extraction with Et₂O (3 mL×3), washing with 0.5 M HCl (2 mL) and brine (2 mL), drying over MgSO₄ (500 mg), filtration and evaporation of all the volatile materials in vacuo to produce the crude epoxy aldehyde. To the crude epoxy aldehyde in THF (3 mL) was added (+)-Ipc2-(Z)-crotylborane 15 (1.0 M in THF, 1.0 mmol) at −78° C. and the resulting solution was stirred at that temperature for 16 hours. After a sequential addition of aqueous NaOH (3.0 M, 1.2 mL) and 30% H₂O₂ (1 mL), normal work-up with EtOAc (4 mL×3) and column chromatography (Et2O/hexane=1/8) rendered the epoxy alcohol 16 and its diastereomer.

H. Synthesis of the Epoxy Alcohol 18

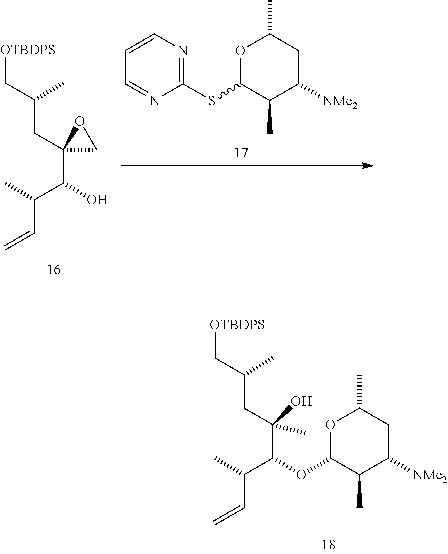

To 16 (1.0 mmol) in THF (3 mL) was added Vitride® (65 wt %/o in toluene, 1.2 mmol) diluted in THF (2 mL) at 0° C. and the mixture was stirred at that temperature for 8 hours. After quenching the reduction with 1 M H₂SO₄ (1 mL), usual work-up with Et2O (3 mL×3) and the following column chromatography (Et₂O/hexane=1/5) imparted the vicinal diol. A heterogeneous mixture of AgOTf (13.1 mmol) and molecular sieve 4 Å (2.1 g) was prepared in a mixture of CH₂Cl₂ (12 mL) and toluene (12 mL). To the heterogeneous mixture were added the vicinal diol (0.87 mmol) in CH₂Cl₂ (6 mL) and 17 (4.35 mmol) in CH₂Cl₂ (6 mL) sequentially at 0° C. The resultant mixture was stirred at 0° C. for 2 hours and then at room temperature for another 2 hours, quenched with saturated aqueous NH₄Cl (15 mL), and filtered through celite (500 mg) with CH₂Cl₂ (10 mL). After separation of the organic layer, the aqueous layer was extracted with EtOAc (5 mL×3), the combined organic layer was dried over MgSO₄ (1 g), filtered and evaporated in vacuo. Chromatographic purification (EtOAc/hexane=1/4) of the crude product provided the 1-glycoside 18 and the starting diol.

I. Synthesis of the Alkene 20

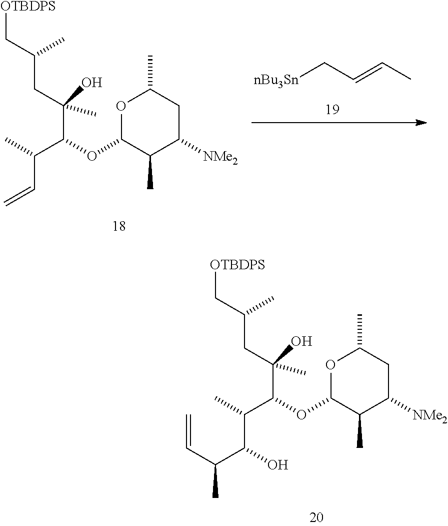

Ozone produced from an ozone generator was bubbled into 18 (0.226 mmol) in MeOH (3 mL) at −78° C. until the starting 18 disappeared completely on TLC. Me₂S (0.2 mL) was added at

−78° C., the reaction temperature was raised to 0° C. and the resulting mixture was stirred at 0° C. for 10 minutes. Evaporation of all the volatile materials under reduced pressure gave rise to the crude aldehyde. To the crude aldehyde in CH₂Cl₂ (11 mL) were added BF₃.OEt₂ (1.36 mmol) and (E)-crotyltin reagent 19 (1.36 mmol) at −78° C. and the mixture was stirred at that temperature for 12 hours. The crotylation was quenched with saturated aqueous NaHCO₃ (9 mL) at −78° C., then with 10% aqueous NaOH (9 mL) at room temperature, and the resultant solution was stirred at that temperature for 12 hours. After normal work-up with CH₂Cl₂ (5 mL×3), the crude product was purified chromatographically two times (EtOAc/hexane=1/3, then Et2O/hexane=1/2) to supply the alkene 20 and presumably its diastereomer.

J. Synthesis of the Hydroxyl Carboxylic Acid 3

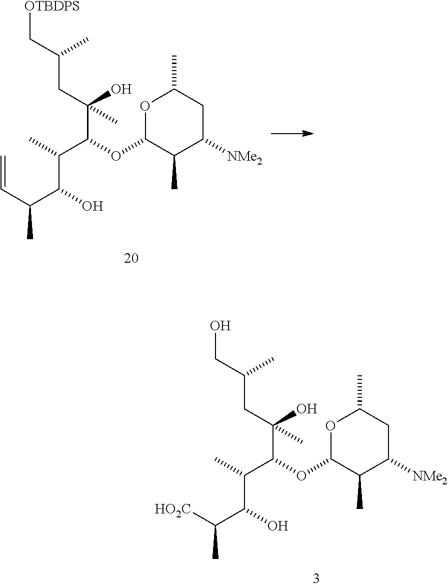

To 20 (0.20 mmol) in DMF (4 mL) were added NaHCO₃ (0.81 mmol), OsO₄ (0.016 mmol) and Oxone® (1.63 mmol) at room temperature, and the mixture was stirred at that temperature for 6 hours. EtOAc (5 mL) and saturated aqueous Na₂S₂O₃ (5 mL) were added and the resulting solution was stirred at room temperature for 20 minutes. After acidifying the solution to pH 3 with 1 M aqueous HCl, usual work-up with EtOAc (3 mL×3) and chromatographic separation (EtOAc/hexane=1/2) procured the silyl protected carboxylic acid. To the silyl protected carboxylic acid (0.17 mmol) in THF (1 mL) was added nBu₄NF (1.0 M in THF, 0.51 mmol) at room temperature and the mixture was stirred at that temperature for 4 hours. Addition of saturated aqueous NH₄Cl (1 mL) followed by normal work-up with CH₂Cl₂ (1 mL×7) and chromatographic purification (MeOH/CH₂Cl₂=1/10) delivered the hydroxyl carboxylic acid 3.

K. Synthesis of the Monoglycosylated Seco-Acid 21

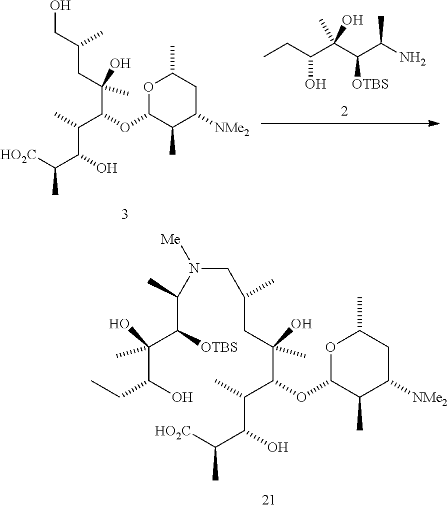

Dess-Martin periodinane (0.27 mmol) was stirred with pyridine (1.10 mmol) in CH₂Cl₂ (1 mL) at room temperature for 15 minutes and 3 (0.22 mmol) in CH₂Cl₂ (0.6 mL) was injected to the periodinane solution cooled down to 0° C. After stirring the reaction mixture at 0° C. for 2 hours, H₂O (2 mL) was added at room temperature and it was worked up with Et₂O (4 mL×4) to offer the crude aldehyde. To a mixture of the crude aldehyde and 2 (0.29 mmol) in MeOH (4 mL) were added NaHCO₃ and 10% Pd/C (11 mg). The reaction flask was briefly evacuated in vacuo and filled with hydrogen gas twice. After 8 hours under an atmospheric pressure of hydrogen gas using a balloon at room temperature, another 10% Pd/C (11 mg) and formalin (37 wt %, 2.23 mmol) were added again, and the mixture was stirred under the hydrogen gas balloon at that temperature for 6 hours more. The resulting solution was filtered through celite (500 mg) with EtOAc (10 mL), the volatile materials were evaporated in vacuo and the remaining residue was purified by column chromatography (EtOAc/hexane=1/1) to produce the seco-acid 21.

L. Synthesis of the Protected Azalide 24

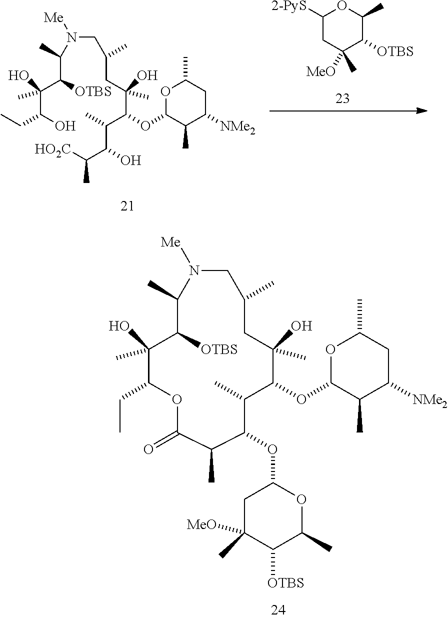

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the cladinoside 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to furnish the protected 1-anomeric azalide 24, the α-anomer and the recovered starting macrolactone.

M. Synthesis of the PP001 1

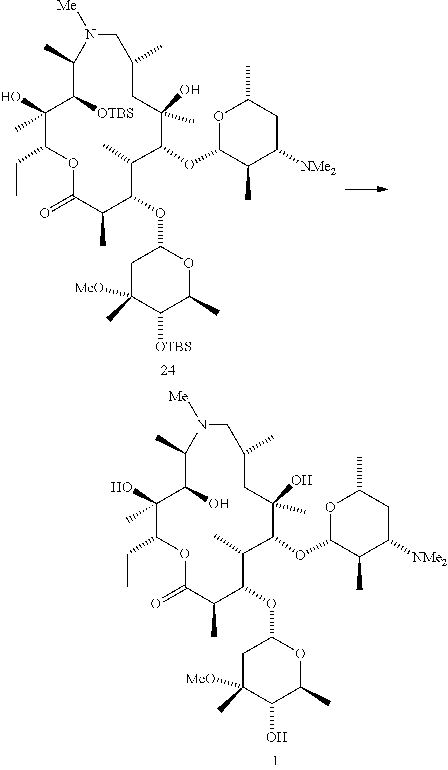

After addition of nBu₄NF (1.0 M in THF, 0.17 mmol) to 24 (0.04 mmol) in THF (0.5 mL) at room temperature, the resulting solution was stirred at that temperature for 5 hours, quenched with saturated aqueous NaHCO₃ (0.5 mL), worked up with EtOAc (1 mL×4) and the crude product was purified by column chromatography (MeOH/CH₂Cl₂=1/8) to yield PP001 1.

### Example 2

### Synthesis of Compound PP002

PP002 is synthesized in 13 steps according to the description below.

A. Synthesis of the benzoate 7

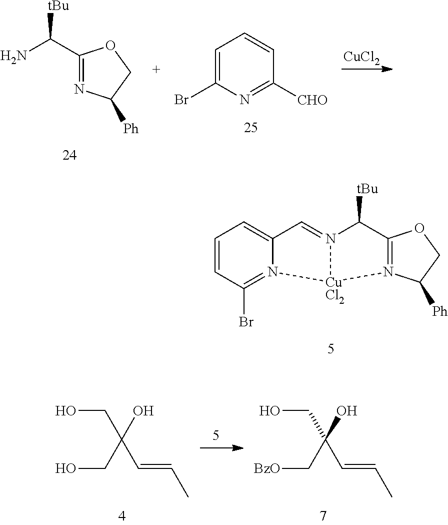

Phenyloxazolineamine 24 (0.40 mmol) in CH₂Cl₂ (5 mL) was reacted with romopyridinecarboxaldehyde 25 (0.40 mmol) in the presence of MgSO₄ (1.99 mmol) at room temperature for 1 hour. Then, CuCl₂ (0.40 mmol) was added and stirred at that temperature for another hour. The mixture as filtered through celite (700 mg) and evaporated in vacuo to generate the catalyst 5. After dissolving the remaining residue in THF (20 mL), the triol 4 (2.00 mmol) in THF (6 mL), Et₃N (2.44 mmol) and benzoyl chloride (2.22 mmol) were added to the catalyst 5 at room temperature in sequence, and then the mixture was stirred at the same temperature for 30 minutes. Quenching the benzoylation with saturated aqueous NH₄Cl (10 mL), work-up with EtOAc (10 mL×3) and the final chromatographic separation (EtOAc/hexane=1/2) produced the monobenzoate 7.

B Synthesis of the Hydroxyl Epoxide 9

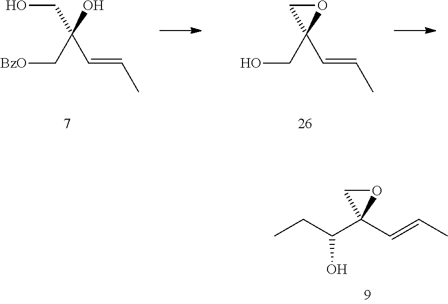

To the benzoate 7 (2.12 mmol) dissolved in CH₂Cl₂ (5 mL) were added MeSO₂Cl (2.58 mmol) and Et₃N (2.72 mmol) at −78° C., and then the mixture was stirred at that temperature for 15 minutes. After raising the reaction temperature to room temperature, DBU (2.57 mmol) in CH₂Cl₂ (1 mL) was injected to the mixture. The resulting solution was stirred for 6 hours at that temperature, and then quenched with saturated aqueous NH₄Cl (3 mL). Normal work-up with CH₂Cl₂ (3 mL×2) and the following column chromatography (Et2O/hexane=1/10) afforded the epoxy benzoate. The epoxy benzoate (1.65 mmol) was dissolved in MeOH (1.5 mL), and subsequently K₂CO₃ (0.25 mmol) was added. After stirring the mixture at room temperature for 2 hours, the reaction was quenched with saturated aqueous NH₄Cl (5 mL). Work-up with CH₂Cl₂ (2 mL×3) and chromatographic purification (EtOAc/hexane=1/5) furnished the epoxy alcohol 26. To 26 (1.47 mmol) in a mixture of DMSO (1 mL) and CH₂Cl₂ (3 mL) were added Et3N (11.76 mmol) and SO₃.Py (11.76 mmol) at 0° C., and the mixture was stirred for 1 hour at that temperature. Work-up was carried out by addition of H₂O (4 mL), extraction with Et₂O (3 mL×3), washing with 0.5 M HCl (2 mL) and brine (2 mL), drying over MgSO₄ (500 mg), filtration, and evaporation of all the volatile materials in vacuo to yield the crude epoxy aldehyde. Et₂Zn (1.0 M in hexane, 2.94 mmol) and the crude aldehyde in toluene (0.5 mL) were sequentially injected to the amino alcohol 8 (16 mg) in toluene (2 mL) at 0° C. After removal of the ice bath, the resulting solution was stirred at room temperature for 24 hours, and then quenched with 1 M HCl (2 mL). Normal work-up with Et₂O (4 mL×3) and the ensuing chromatographic separation (Et₂O/hexane=1/7) gave the epoxy alcohol 9 and its diastereomer.

C. Synthesis of the Epoxide 10

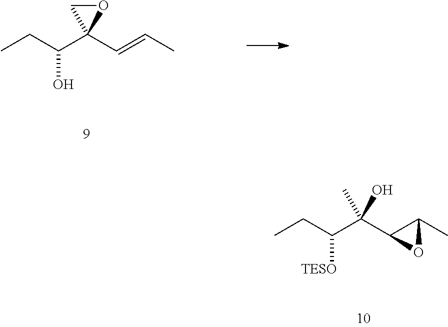

To 9 (2.11 mmol) in THF (2 mL) was added Vitride® (65 wt % in toluene, 2.53 mmol) diluted in THF (2 mL) at 0° C. and the mixture was stirred at that temperature for 8 hours. After quenching the reduction with 1 M H₂SO₄ (2 mL), usual work-up with Et2O (3 mL×3), and the following column chromatography (Et₂O/hexane=1/3) provided the diol. Triethylsilyl chloride (2.21 mmol) and imidazole (2.80 mmol) were added to the diol (1.86 mmol) in DMF (2 mL) at room temperature in sequence and the resulting solution was stirred at that temperature for 8 hours. The silylation was quenched with H₂O (2 mL), work-up with Et₂O (3 mL×3) and the crude product was separated chromatographically (EtOAc/hexane=1/8) to render the TES ether. To the TES ether (1.75 mmol) in CH₂Cl₂ (4 mL) was added m-chloroperbenzoic acid (77% purity, 2.63 mmol) at −50° C. and the mixture was stirred at that temperature for 6 hours. After quenching the epoxidation with 1 M aqueous NaOH (2 mL), usual work-up with EtOAc (3 mL×3) and chromatographic purification (EtOAC/hexane=1/6) gave rise to the silyloxy epoxide 10.

D. Synthesis of the amine 2

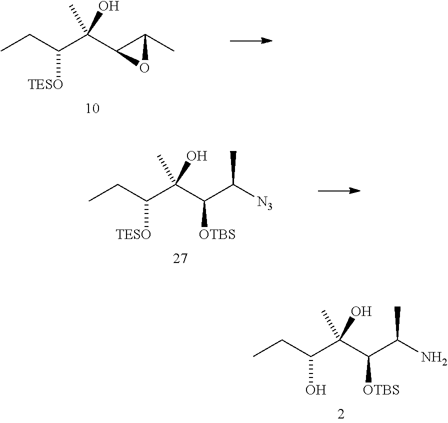

NaN₃ (3.64 mmol) and MgSO₄ (3.64 mmol) were added to 10 (1.82 mmol) in 2-methoxyethanol (5 mL) at room temperature, and the resulting mixture was heated at 11° C. for 6 hours. After cooling the mixture to room temperature, it was filtered through celite (500 mg) with EtOAc (10 mL). The filtrate was evaporated in vacuo and the residue was separated chromatographically (EtOAc/hexane=1/4) to impart the hydroxyl azide. To the hydroxyl azide (1.49 mmol) in DMF (4 mL) were added t-butyldimethylsilyl chloride (2.10 mmol) and imidazole (2.38 mmol) at room temperature for 5 hours. Quenching the silylation with H₂O (2 mL), work-up with Et₂O (4 mL×3) and column chromatography (Et₂O/hexane=1/30) supplied the TBS ether azide 27. Ph₃P (2.68 mmol) was added to 27 (1.34 mmol) in a 10:1 mixture of THF and H₂O (4.4 mL) at room temperature, and the solution was stirred at that temperature for 10 hours. After evaporation of the volatile materials in vacuo, the residue was purified chromatographically (Et₂O/hexane=1/10) to procure the silyl ether amine. Pyridinium fluoride in a mixture of THF (6 mL) and pyridine (60 μL) was injected to the silyl ether amine (1.17 mmol) in THF (2 mL) at 0° C., and the mixture was stirred at room temperature for 5 hours. After addition of saturated aqueous NaHCO₃ (2 mL), normal work-up with EtOAc (3 mL×3) and column chromatography (EtOAc/hexane=1/2) delivered the amine 2.

E. Synthesis of the Triol 13

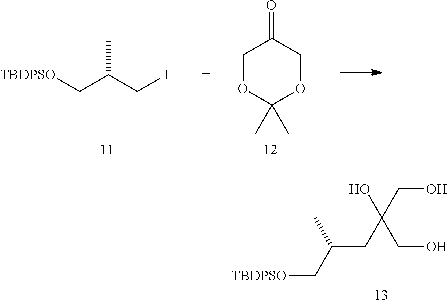

To a mixture of the iodide 11 (3.07 mmol) and the ketone 12 (3.07 mmol) in THF (16 mL) was added s-BuLi (1.4 M in cyclohexane, 5.53 mmol) dropwise at −98° C. The reaction solution was stirred at −98° C. for 2 hours and then quenched with saturated aqueous NH₄Cl (10 mL). Normal work-up with Et₂O (5 mL×3) and chromatographic separation (Et₂O/hexane=1/10) offered the adduct acetonide. Propane-1,3-dithiol (5.03 mmol) and BF₃OEt₂ (0.12 mmol) were added to the adduct acetonide (1.93 mmol) in CH₂Cl₂ (5 mL) at 0° C., and then the mixture was stirred at 0° C. for 1 hour. Quenching the hydrolysis with saturated aqueous NaHCO₃ (3 mL), work-up with EtOAc (4 mL×3) and column chromatography (MeOH/CH₂Cl₂=1/15) afforded the triol 13.

F. Synthesis of the Monobenzoate 14

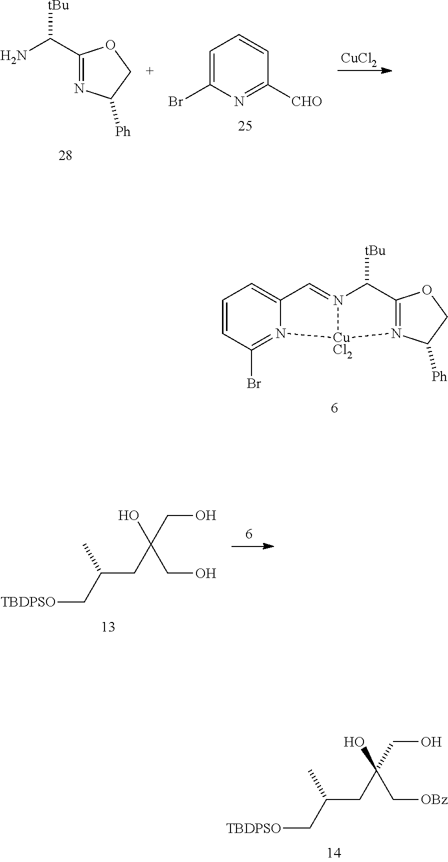

Phenyloxazolineamine 28 (0.20 mmol) in CH₂Cl₂ (3 mL) was reacted with bromopyridinecarboxaldehyde 25 (0.20 mmol) in the presence of MgSO₄ (0.99 mmol) at room temperature for 1 hour. Then, CuCl₂ (0.20 mmol) was added and stirred at that temperature for another hour. The mixture was filtered through celite (400 mg) and evaporated in vacuo to generate the catalyst 6. After dissolving the remaining residue in THF (10 mL) the triol 13 (1.00 mmol) in THF (3 mL), Et₃N (1.20 mmol) and benzoyl chloride (1.10 mmol) were added to the catalyst 6 at room temperature in sequence, and then the mixture was stirred at the same temperature for 30 minutes. Quenching the benzoylation with saturated aqueous NH₄Cl (5 mL), work-up with EtOAc (5 mL×3) and the following chromatographic separation (EtOAc/hexane=1/4) produced the monobenzoate 14 and its diastereomeric monobenzoate.

G. Synthesis of the Epoxy Alcohol 16

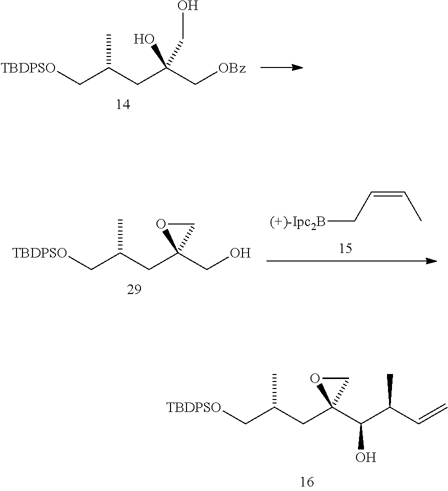

To the monobenzoate 14 (1.38 mmol) in CHBCl₂ (5 mL) were added MeSO₂Cl (1.67 mmol) and Et₃N (1.80 mmol) at −78° C., and then the mixture was stirred at that temperature for 30 minutes. After raising the reaction temperature to room temperature, DBU (1.66 mmol) in CH₂Cl₂ (1 mL) was injected to the mixture. The resulting solution was stirred for 6 hours at that temperature, and then quenched with saturated aqueous NH₄Cl (3 mL). Normal work-up with CH₂Cl₂ (3 mL×3) and the following column chromatography (Et₂O/hexane=1/15) yielded the epoxy benzoate. The epoxy benzoate (1.08 mmol) was dissolved in MeOH (3 mL), and subsequent K₂CO₃ (0.16 mmol) was added. After stirring the mixture at room temperature for 2 hours, the reaction was quenched with saturated aqueous NH₄Cl (2 mL). Work-up with CH₂Cl₂ (2 mL×3) and chromatographic purification (EtOAc/hexane=1/9) gave the epoxy alcohol 29. To 29 (0.97 mmol) in a mixture of DMSO (1 mL) and CH₂Cl₂ (3 mL) were added Et₃N (1.1 mL, 7.79 mmol) and SO₃Py (7.78 mmol) at 0° C., and the mixture was stirred for 1 hour at that temperature. Work-up was carried out by addition of H₂O (4 mL), extraction with Et₂O (3 mL×3), washing with 0.5 M HCl (2 mL) and brine (2 mL), drying over MgSO₄ (500 mg), filtration and evaporation of all the volatilematerials in vacuo to produce the crude epoxy aldehyde. To the crude epoxy aldehyde in THF (3 mL) was added (+)-Ipc2-(Z)-crotylborane 15 (1.0 M in THF, 1.0 mmol) at −78° C. and the resulting solution was stirred at that temperature for 16 hours. After a sequential addition of aqueous NaOH (3.0 M, 1.2 mL) and 30% H₂O₂ (1 mL), normal work-up with EtOAc (4 mL×3) and column chromatography (Et2O/hexane=1/8) rendered the epoxy alcohol 16 and its diastereomer.

H. Synthesis of the Epoxy Alcohol 18

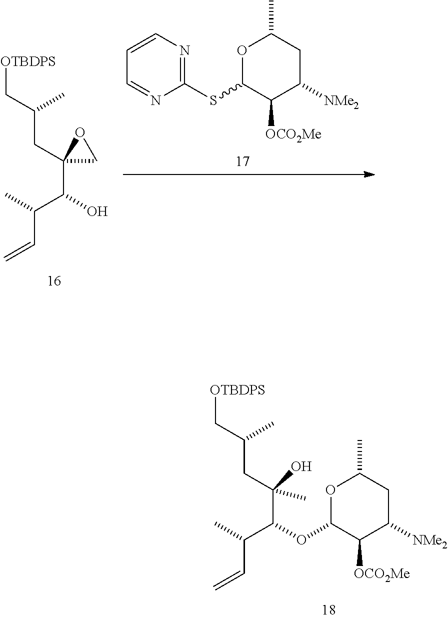

To 16 (1.0 mmol) in THF (3 mL) was added Vitride® (65 wt % in toluene, 1.2 mmol) diluted in THF (2 mL) at 0° C. and the mixture was stirred at that temperature for 8 hours. After quenching the reduction with 1 M H₂SO₄ (1 mL), usual work-up with Et2O (3 mL×3) and the following column chromatography (Et₂O/hexane=1/5) imparted the vicinal diol. A heterogeneous mixture of AgOTf (13.1 mmol) and molecular sieve 4 Å (2.1 g) was prepared in a mixture of CH₂Cl₂ (12 mL) and toluene (12 mL). To the heterogeneous mixture were added the vicinal diol (0.87 mmol) in CH₂Cl₂ (6 mL) and the desosaminating agent 17 (4.35 mmol) in CH₂Cl₂ (6 mL) sequentially at 0° C. The resultant mixture was stirred at 0° C. for 2 hours and then at room temperature for another 2 hours, quenched with saturated aqueous NH₄Cl (15 mL), and filtered through celite (500 mg) with CH₂Cl₂ (10 mL). After separation of the organic layer, the aqueous layer was extracted with EtOAc (5 mL×3), the combined organic layer was dried over MgSO₄ (1 g), filtered and evaporated in vacuo. Chromatographic purification (EtOAc/hexane=1/4) of the crude product provided the 1-glycoside 18 and the starting diol.

I. Synthesis of the Alkene 20

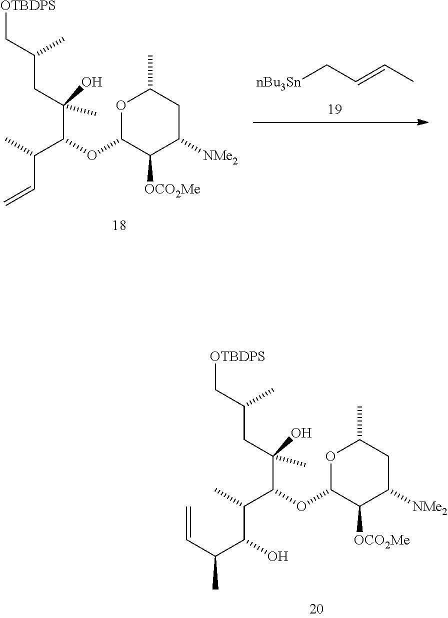

Ozone produced from an ozone generator was bubbled into 18 (0.226 mmol) in MeOH (3 mL) at −78° C. until the starting 18 disappeared completely on TLC. Me₂S (0.2 mL) was added at −78° C., the reaction temperature was raised to 0° C. and the resulting mixture was stirred at 0° C. for 10 minutes. Evaporation of all the volatile materials under reduced pressure gave rise to the crude aldehyde. To the crude aldehyde in CH₂Cl₂ (11 mL) were added BF₃OEt₂ (1.36 mmol) and (E)-crotyltin reagent 19 (1.36 mmol) at −78° C. and the mixture was stirred at that temperature for 12 hours. The crotylation was quenched with saturated aqueous NaHCO₃ (9 mL) at −78° C., then with 10% aqueous NaOH (9 mL) at room temperature, and the resultant solution was stirred at that temperature for 12 hours. After normal work-up with CH₂Cl₂ (5 mL×3), the crude product was purified chromatographically two times (EtOAc/hexane=1/3, then Et2O/hexane=1/2) to supply the alkene 20 and presumably its diastereomers.

J. Synthesis of the Hydroxyl Carboxylic Acid 3

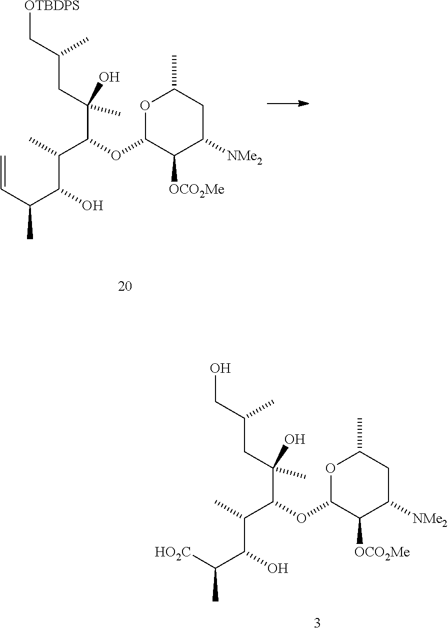

To 20 (0.20 mmol) in DMF (4 mL) were added NaHCO₃ (0.81 mmol), OsO₄ (0.016 mmol) and Oxone® (1.63 mmol) at room temperature, and the mixture was stirred at that temperature for 6 hours. EtOAc (5 mL) and saturated aqueous Na₂S₂O₃ (5 mL) were added and the resulting solution was stirred at room temperature for 20 minutes. After acidifying the solution to pH 3 with 1 M aqueous HCl, usual work-up with EtOAc (3 mL×3) and chromatographic separation (EtOAc/hexane=1/2) procured the silyl protected carboxylic acid. To the silyl protected carboxylic acid (0.17 mmol) in THF (1 mL) was added nBu₄NF (1.0 M in THF, 0.51 mmol) at room temperature and the mixture was stirred at that temperature for 4 hours. Addition of saturated aqueous NH₄Cl (1 mL) followed by normal work-up with CH₂Cl₂ (1 mL×7) and chromatographic purification (MeOH/CH₂Cl₂=1/10) delivered the hydroxyl carboxylic acid 3.

K. Synthesis of 21

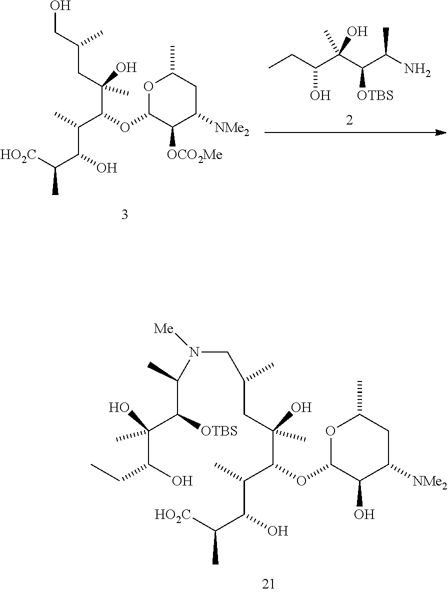

Dess-Martin periodinane (0.27 mmol) was stirred with pyridine (1.10 mmol) in CH₂Cl₂ (1 mL) at room temperature for 15 minutes and 3 (0.22 mmol) in CH₂Cl₂ (0.6 mL) was injected to the periodinane solution cooled down to 0° C. After stirring the reaction mixture at 0° C. for 2 hours, H₂O (2 mL) was added at room temperature and it was worked up with Et₂O (4 mL×4) to offer the crude aldehyde. To a mixture of the crude aldehyde and 2 (0.29 mmol) in MeOH (4 mL) were added NaHCO₃ and 10% Pd/C (11 mg). The reaction flask was briefly evacuated in vacuo and filled with hydrogen gas twice. After 8 hours under an atmospheric pressure of hydrogen gas using a balloon at room temperature, another 10% Pd/C (11 mg) and formalin (37 wt %/o, 2.23 mmol) were added again, and the mixture was stirred under the hydrogen gas balloon at that temperature for 6 hours more. The resulting solution was filtered through celite (500 mg) with EtOAc (10 mL), the volatile materials were evaporated in vacuo and the remaining residue was purified by column chromatography (EtOAc/hexane=1/1) to produce the seco-acid 21.

L. Synthesis of the Protected Azalide 24

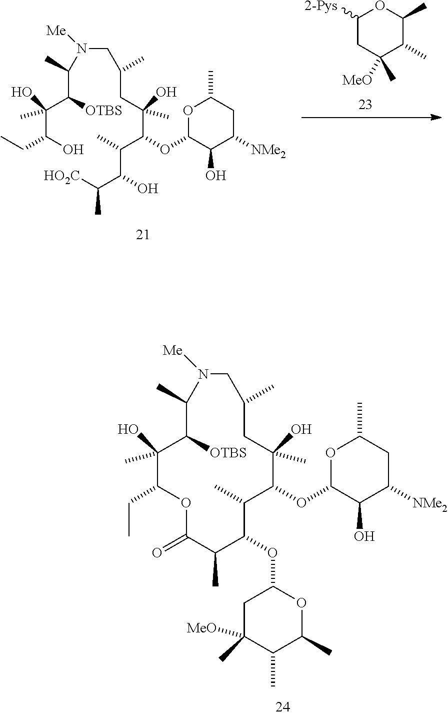

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to furnish the 1-anomeric azalide 24, the α-anomer and the recovered starting macrolactone.

M. Synthesis of the PP02 1

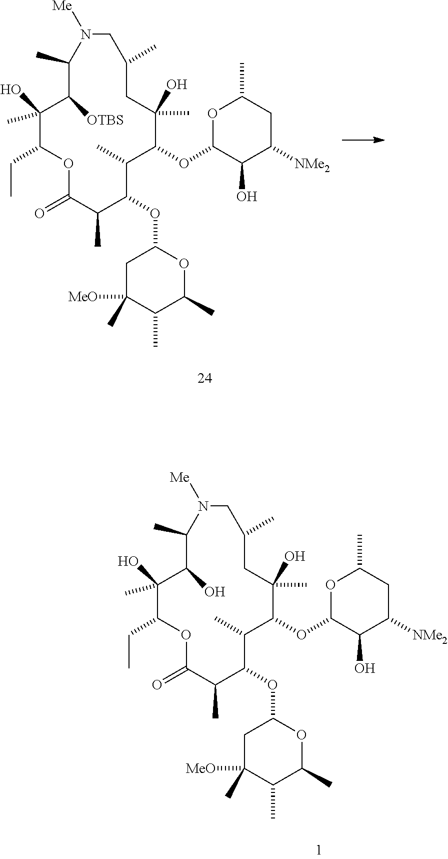

After addition of nBu₄NF (1.0 M in THF, 0.17 mmol) to 24 (0.04 mmol) in THF (0.5 mL) at room temperature, the resulting solution was stirred at that temperature for 5 hours, quenched with saturated aqueous NaHCO₃ (0.5 mL), worked up with EtOAc (1 mL×4) and the crude product was purified by column chromatography (MeOH/CH₂Cl₂=1/8) to yield PP002 1

### Example 3

### Synthesis of Compound PP003

PP003 is synthesized according to the synthesis of PP001 step A to K. Step L is described below.

L. Synthesis of PP003 24

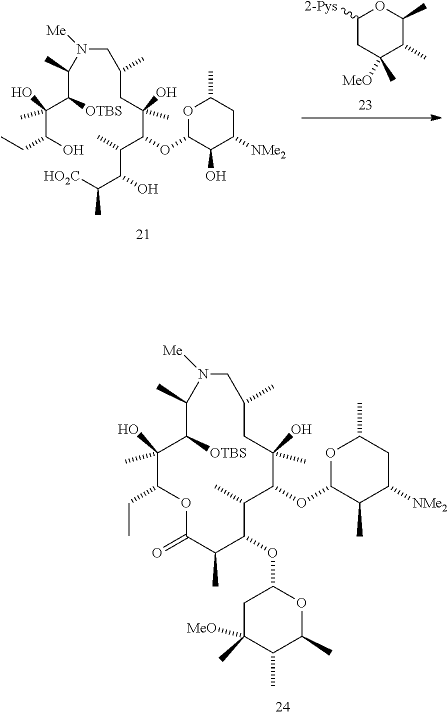

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to produce PP003 24.

### Example 4

### Synthesis of Compound PP004

PP004 is synthesized in 13 steps. Step A to K and M is performed according to the synthesis of PP002 and the step L is modified according to the description below.

L. Synthesis of 24

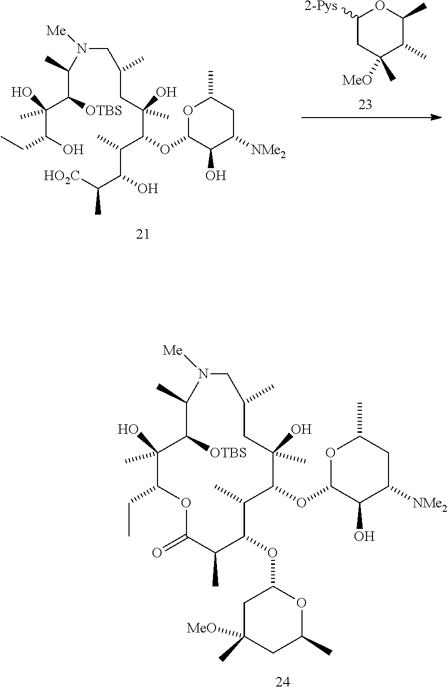

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to furnish the 3-anomeric azalide 24, the α-anomer and the recovered starting macrolactone.

M. Synthesis of the PP004 1

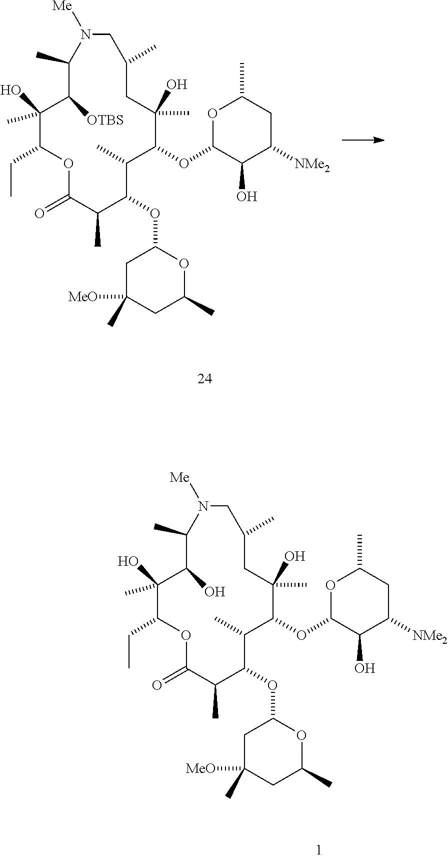

After addition of nBu₄NF (1.0 M in THF, 0.17 mmol) to 24 (0.04 mmol) in THF (0.5 mL) at room temperature, the resulting solution was stirred at that temperature for 5 hours, quenched with saturated aqueous NaHCO₃ (0.5 mL), worked up with EtOAc (1 mL×4) and the crude product was purified by column chromatography (MeOH/CH₂Cl₂=1/8) to yield PP004 1

### Example 5

### Synthesis of Compound PP005

PP005 is synthesized in 13 steps. Step A to G is performed as described for PP001. In step H the reactant 17 is changed giving rise to PP005. The synthesis form step H is described below.

Synthesis

Step A to G according to PP001.

H. Synthesis of the Epoxy Alcohol 18

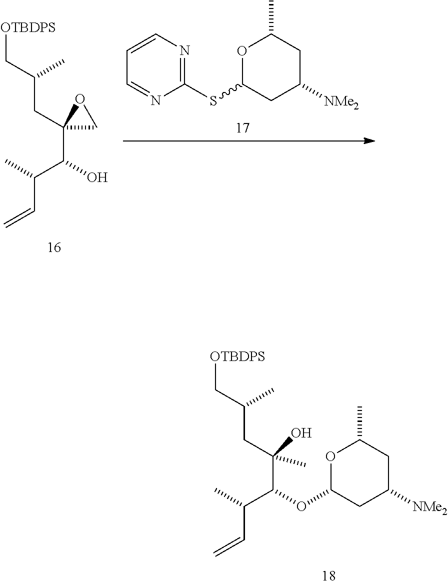

To 16 (1.0 mmol) in THF (3 mL) was added Vitride® (65 wt % in toluene, 1.2 mmol) diluted in THF (2 mL) at 0° C. and the mixture was stirred at that temperature for 8 hours. After quenching the reduction with 1 M H₂SO₄ (1 mL), usual work-up with Et₂O (3 mL×3) and the following column chromatography (Et₂O/hexane=1/5) imparted the vicinal diol. A heterogeneous mixture of AgOTf (13.1 mmol) and molecular sieve 4 Å (2.1 g) was prepared in a mixture of CH₂Cl₂ (12 mL) and toluene (12 mL). To the heterogeneous mixture were added the vicinal diol (0.87 mmol) in CH₂Cl₂ (6 mL) and 17 (4.35 mmol) in CH₂Cl₂ (6 mL) sequentially at 0° C. The resultant mixture was stirred at 0° C. for 2 hours and then at room temperature for another 2 hours, quenched with saturated aqueous NH₄Cl (15 mL), and filtered through celite (500 mg) with CH₂Cl₂ (10 mL). After separation of the organic layer, the aqueous layer was extracted with EtOAc (5 mL×3), the combined organic layer was dried over MgSO₄ (1 g), filtered and evaporated in vacuo. Chromatographic purification (EtOAc/hexane=1/4) of the crude product provided the β-glycoside 18 and the starting diol.

I. Synthesis of the alkene 20

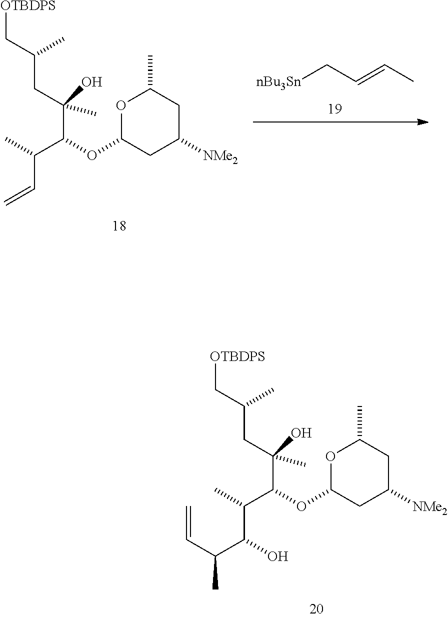

Ozone produced from an ozone generator was bubbled into 18 (0.226 mmol) in MeOH (3 mL) at −78° C. until the starting 18 disappeared completely on TLC. Me₂S (0.2 mL) was added at −78° C., the reaction temperature was raised to 0° C. and the resulting mixture was stirred at 0° C. for 10 minutes. Evaporation of all the volatile materials under reduced pressure gave rise to the crude aldehyde. To the crude aldehyde in CH₂Cl₂ (11 mL) were added BF₃.OEt₂ (1.36 mmol) and (E)-crotyltin reagent 19 (1.36 mmol) at −78° C. and the mixture was stirred at that temperature for 12 hours. The reaction was quenched with saturated aqueous NaHCO₃ (9 mL) at −78° C., then with 10% aqueous NaOH (9 mL) at room temperature, and the resultant solution was stirred at that temperature for 12 hours. After normal work-up with CH₂Cl₂ (5 mL×3), the crude product was purified chromatographically two times (EtOAc/hexane=1/3, then Et2O/hexane=1/2) to supply 20 and presumably its diastereomer.

J. Synthesis of 3

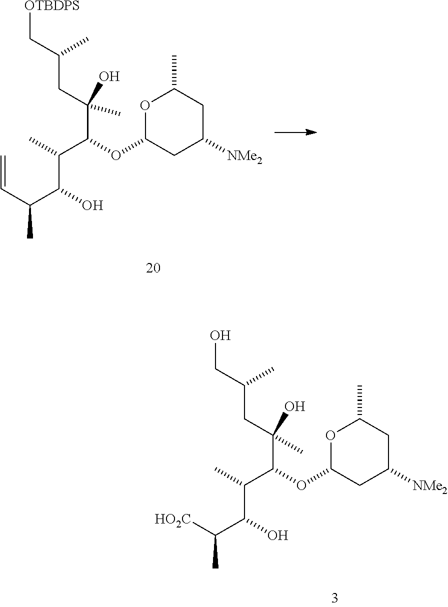

To 20 (0.20 mmol) in DMF (4 mL) were added NaHCO₃ (0.81 mmol), OsO₄ (0.016 mmol) and Oxone® (1.63 mmol) at room temperature, and the mixture was stirred at that temperature for 6 hours. EtOAc (5 mL) and saturated aqueous Na₂S₂O₃ (5 mL) were added and the resulting solution was stirred at room temperature for 20 minutes. After acidifying the solution to pH 3 with 1 M aqueous HCl, usual work-up with EtOAc (3 mL×3) and chromatographic separation (EtOAc/hexane=1/2) procured the silyl protected carboxylic acid. To the silyl protected carboxylic acid (0.17 mmol) in THF (1 mL) was added nBu₄NF (1.0 M in THF, 0.51 mmol) at room temperature and the mixture was stirred at that temperature for 4 hours. Addition of saturated aqueous NH₄Cl (1 mL) followed by normal work-up with CH₂Cl₂ (1 mL×7) and chromatographic purification (MeOH/CH₂Cl₂=1/10) delivered 3.

K. Synthesis of 21

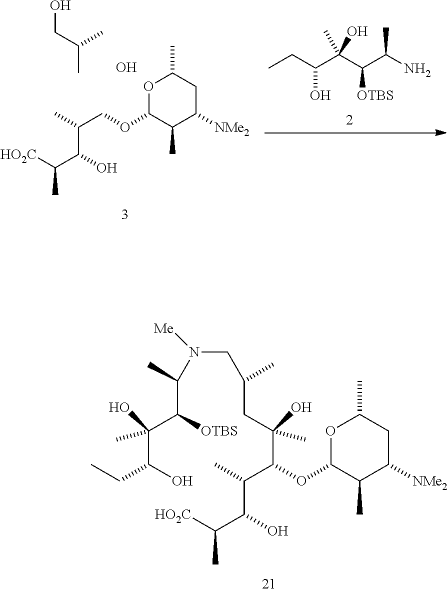

Dess-Martin periodinane (0.27 mmol) was stirred with pyridine (1.10 mmol) in CH₂Cl₂ (1 mL) at room temperature for 15 minutes and 3 (0.22 mmol) in CH₂Cl₂ (0.6 mL) was injected to the periodinane solution cooled down to 0° C. After stirring the reaction mixture at 0° C. for 2 hours, H₂O (2 mL) was added at room temperature and it was worked up with Et₂O (4 mL×4) to offer the crude product. To a mixture of the crude product and 2 (0.29 mmol) in MeOH (4 mL) were added NaHCO₃ and 10% Pd/C (11 mg). The reaction flask was briefly evacuated in vacuo and filled with hydrogen gas twice. After 8 hours under an atmospheric pressure of hydrogen gas using a balloon at room temperature, another 10% Pd/C (11 mg) and formalin (37 wt %/o, 2.23 mmol) were added again, and the mixture was stirred under the hydrogen gas balloon at that temperature for 6 hours more. The resulting solution was filtered through celite (500 mg) with EtOAc (10 mL), the volatile materials were evaporated in vacuo and the remaining residue was purified by column chromatography (EtOAc/hexane=1/1) to produce 21.

L. Synthesis of the Protected Azalide 24

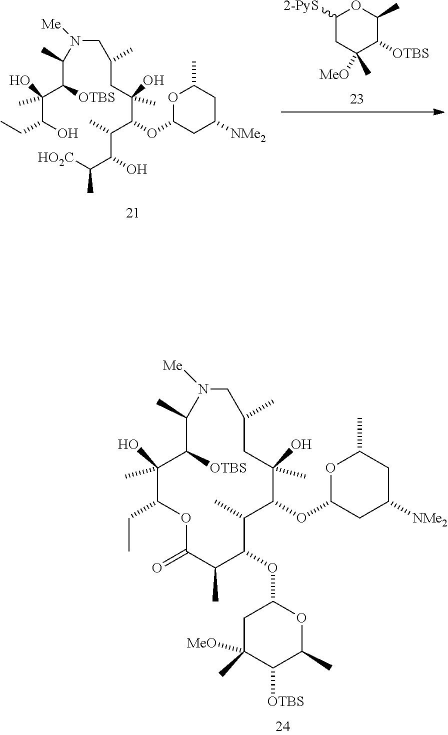

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the cladinoside 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to furnish the protected 1-anomeric azalide 24, the α-anomer and the recovered starting macrolactone.

M. Synthesis of the PP005 1

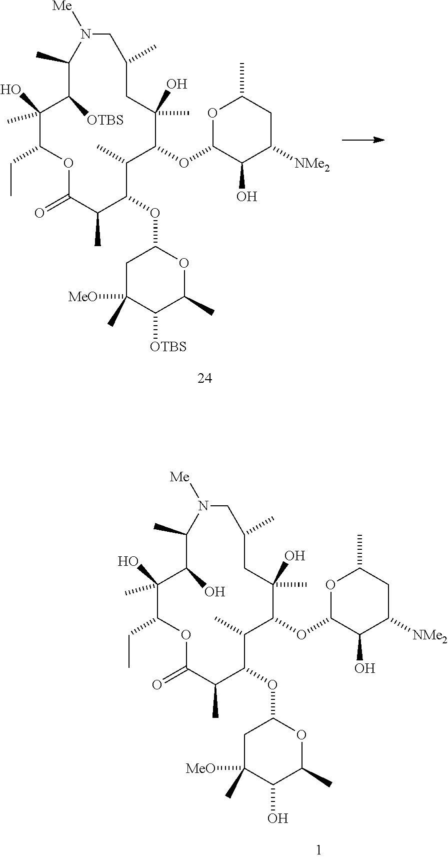

After addition of nBu₄NF (1.0 M in THF, 0.17 mmol) to 24 (0.04 mmol) in THF (0.5 mL) at room temperature, the resulting solution was stirred at that temperature for 5 hours, quenched with saturated aqueous NaHCO₃ (0.5 mL), worked up with EtOAc (1 mL×4) and the crude product was purified by column chromatography (MeOH/CH₂Cl₂=1/8) to yield PP005 1

### Example 6

### Synthesis of Compound PP006

PP006 is synthesized in 13 steps. Step A to K is performed as described for PP005 and step L as described below.

Step A to K According to PP005.

L. Synthesis of PP006

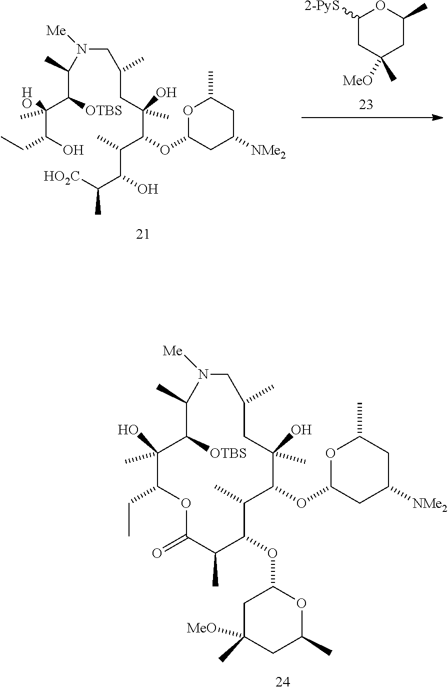

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the cladinoside 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂CH₂=1/20) to produce PP006 24

### Example 7

### Synthesis of Compound PP007

PP007 is synthesized in 12 steps. Step A to K is performed as described for PP003. In step L the reactant 23 is changed. The synthesis form step L is described below.

Step A to K According to PP003.

L. Synthesis of PP007 24

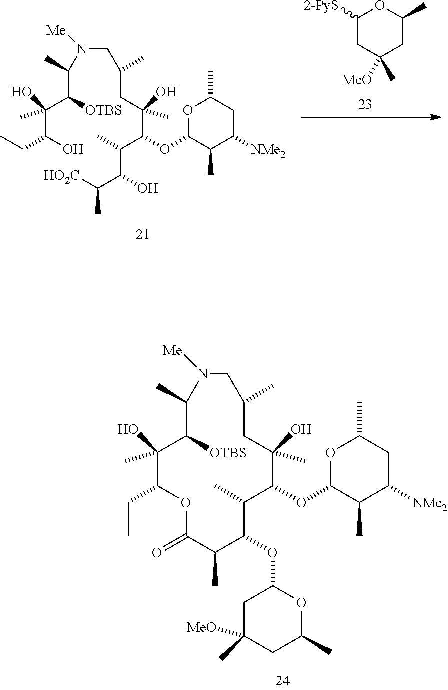

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂CH₂=1/20) to produce PP007 24.

### Example 8

### Synthesis of Compound PP008

PP008 is synthesized in 12 steps. Step A to K is performed as described for PP008. In step L the reactant 23 is changed. The synthesis form step L is described below.

Step A to K According to PP006.

L. Synthesis of PP008 24

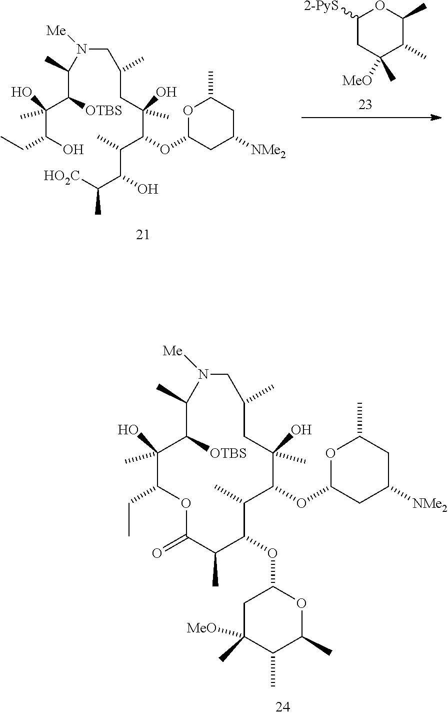

To 21 (0.07 mmol) in toluene (15 mL) were added 2,4,6-trichlorobenzoyl chloride (0.21 mmol), Et₃N (0.42 mmol) and 4(dimethylamino)pyridine (0.06 mmol) at room temperature. After stirring the mixture at that temperature for 1 hour, it was quenched with saturated aqueous NaHCO₃ (3 mL), worked up with EtOAc (4 mL×3) and the crude product was separated chromatographically (acetone/CH₂Cl₂=1/15) to afford the macrolactone 22. To a mixture of the macrolactone 22 (0.06 mmol) and the 23 (0.48 mmol) were added CuO (2.17 mmol), molecular sieve 4 Å (800 mg), acetonitrile (3 mL) and cupic trifluoromethanesulfonate (0.96 mmol) in sequence at room temperature, and the mixture was stirred at that temperature for 3 hours. The glycosylation was quenched with saturated aqueous NaHCO₃ (3 mL) and the resulting solution was filtered through celite (500 mg) using EtOAc (10 mL). After separation of the organic layer, the aqueous layer was extracted with EtOAc (2 mL×3), the combined organic layer was dried with MgSO₄ (300 mg), filtered, evaporated in vacuo and the remaining residue was purified by column chromatography (acetone/CH₂Cl₂=1/20) to produce PP008 24.

### Example 9

### Antimicrobial Disk Susceptibility Test

A test for the antimicrobial activity of the novel compounds were performed according to the standards of Clinical and Laboratory Standards Institute, Performance Standards for Antimicrobial Disk Susceptibility Tests; approved Standard; M2-A), vol. 26 NO. 1 9^{th} ed. The samples were dissolved in 10 ml of sterile Milli-Q water by magnetic stirring overnight at 20° C.

TABLE 1
Antimicrobial Disk Susceotibility Tests

| | | S. | | E. | | P. | | K. | |
|---|---|---|---|---|---|---|---|---|---|
| | Dose | aureus | | coli | | aeruginosa | | pneumonia | |
| Sample | (μg/disk) | a) | b) | a) | b) | a) | b) | a) | b) |
| PP001 | 10 | <1 | <1 | <1 | <1 | <1 | <1 | <1 | <1 |
| PP001 | 5 | <1 | <1 | <1 | <1 | <1 | <1 | <1 | <1 |
| PP002 | 10 | <1 | <1 | <1 | <1 | <1 | <1 | <1 | <1 |
| PP002 | 5 | <1 | <1 | <1 | <1 | <1 | <1 | <1 | <1 |
| PP003 | 10 | <1 | <1 | <1 | <1 | <1 | <1 | <1 | <1 |
| PP003 | 5 | <1 | <1 | <1 | <1 | <1 | <1 | <1 | <1 |
| PP004 | 10 | <1 | <1 | <1 | <1 | <1 | <1 | <1 | <1 |
| PP004 | 5 | <1 | <1 | <1 | <1 | <1 | <1 | <1 | <1 |
| PP005 | 10 | <1 | <1 | <1 | <1 | <1 | <1 | <1 | <1 |
| PP005 | 5 | <1 | <1 | <1 | <1 | <1 | <1 | <1 | <1 |
| PP006 | 10 | <1 | <1 | <1 | <1 | <1 | <1 | <1 | <1 |
| PP006 | 5 | <1 | <1 | <1 | <1 | <1 | <1 | <1 | <1 |
| PP007 | 10 | <1 | <1 | <1 | <1 | <1 | <1 | <1 | <1 |
| PP007 | 5 | <1 | <1 | <1 | <1 | <1 | <1 | <1 | <1 |
| PP008 | 10 | <1 | <1 | <1 | <1 | <1 | <1 | <1 | <1 |
| PP008 | 5 | <1 | <1 | <1 | <1 | <1 | <1 | <1 | <1 |

| | | | | | |
|---|---|---|---|---|---|
| Control | — | <1 | <1 | <1 | <1 |
| Gentamicin | 10 | 30.4 | 27.9 | 27.2 | 24.7 |

S. aureus: Staphylococcus aureus ATTC 6538
E. coli: Escherichia coli ATCC 8739
P. aeruginosa: Pseudomonas aeruginosa ATCC 9027
K. pneumonia: Klebsiella pneumonia ATCC 35657

Two doses of the samples were tested in duplicate, a) and b). The size of the inhibition zones were measured in mm after incubation. It was found that all samples, PP001-8, had no antibiotic activity when tested against 4 different microorganisms.

### Example 10

### Azithromycin Effect on the Respiratory Function

The compounds according to the present invention, such as PP001-PP008, is expected to show a similar result regarding azithromycin's non-antibiotic properties when these are tested on human lung cells for processing on tight junction proteins claudin-1, claudin-4, occludin and JAM-A and how they affect the cells transepithelial electrical resistance (TER) assays as a measure for strengthened intercellular epithelial coherence, or immunomodulating assays, or any of the methods applied in references 1, 2, 3, 4, 5 or 6.

It will be observed that the tested compounds of the present invention maintain most of their non-antibiotic properties.

As these compounds do not show any significant or a lower antibiotic activity, it makes them suitable to use for medical purposes.

### REFERENCES

1. Keicho, N., and S. Kudoh. 2002. Diffuse panbronchiolitis: role of macrolides in therapy. Am. J. Respir. Med. 1:119-131.
2. Schultz, M. J. 2004. Macrolide activities beyond their antimicrobial effects: macrolides in diffuse panbronchiolitis and cystic fibrosis. J. Antimicrob. Chemother. 54:21-28
3. Equi, A., I. M. Balfour-Lynn, A. Bush, and M. Rosenthal. 2002. Long term azithromycin in children with cystic fibrosis: a randomised, placebo-controlled crossover trial. Lancet 360:978-984.
4. Saiman, L., B. C. Marshall, N. Mayer-Hamblett, J. L. Burns, A. L. Quittner, D. A. Cibene, S. Coquillette, A. Y. Fieberg, F. J. Accurso, and P. W. Campbell III. 2003. Azithromycin in patients with cystic fibrosis chronically infected with Pseudomonas aeruginosa: a randomized controlled trial. JAMA 290: 1749-1756. 19. Schneeberge
5. Wolter, J., S. Seeney, S. Bell, S. Bowler, P. Masel, and J. McCormack. 2002. Effect of long term treatment with azithromycin on disease parameters in cystic fibrosis: a randomised trial. Thorax 57:212-216.
6. Asgrimsson, V., et al, Novel effect of azilthromycin on tight junction proteins in human airway epithelia. Antimicrobial Agents and Chemotheraphy, May 2006, pp. 1805-1812.

What is claimed is:

1. A compound of Formula (I) 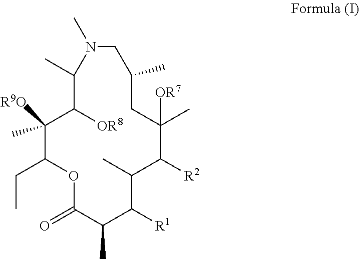
wherein
R¹ is OH, CH₃, OCH₃, a C₂-C₄ straight or branched alkyl group, or the group R³, which is bounded to Formula (I) via a covalent bonding to oxygen, where R⁵ is H, OH or CH₃, 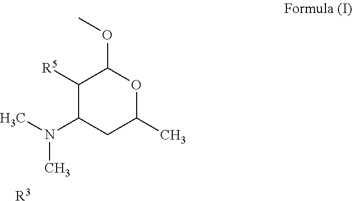
R² is OH, CH₃, OCH₃, a C₂-C₄ straight or branched alkyl group, or the group R⁴, which is bounded to Formula (I) via a covalent bonding to oxygen, where R⁶ is H, OH or CH₃, 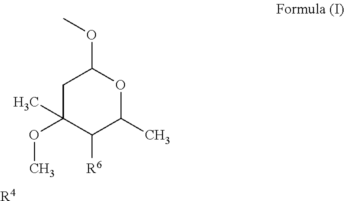
R⁷ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
R⁸ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
R⁹ is hydrogen, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C5- or C6-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine, halogen is Cl, Br, or I,
or a pharmaceutically derivative thereof, tautomers and stereoisomers thereof, or a pharmaceutically acceptable salt thereof, and
with the provisio that
R⁵ and R⁶ cannot both be OH.

2. The compound of claim 1, wherein
R¹ is the group R³, where R⁵ is H, OH or CH₃, and
R² is OH, CH₃, or OCH₃.

3. The compound of claim 1, wherein
R¹ is OH, CH₃, OCH₃, and R² is the group R⁴, with R⁶ being H, OH or CH₃.

4. The compound of claim 1, selected from the group consisting of
i) compound of Formula (I) wherein R¹ is the group R³, with R⁵ being CH₃, and R² is the group R⁴, with R⁶ being OH;
ii) compound of Formula (I) wherein R¹ is the group R³, with R⁵ being OH and R² is the group R⁴, with R⁶ being CH₃;
iii) compound of Formula (I) wherein R¹ is the group R³, with R⁵ being CH₃ and R² is the group R⁴, with R⁶ being CH₃;
iv) compound of Formula (I) wherein R¹ is the group R³, with R⁵ being OH and R² is the group R⁴, with R⁶ being H;
v) compound of Formula (I) wherein R¹ is the group R³, with R⁵ being H and R² is the group R⁴, with R⁶ being OH;
vi) compound of Formula (I) wherein R¹ is the group R³, with R⁵ being H and R² is the group R⁴, with R⁶ being H;
vii) compound of Formula (I) wherein R¹ is the group R³, with R⁵ being CH₃ and R² is the group R⁴, with R⁶ being H;
viii) compound of Formula (I) wherein R¹ is the group R³, with R⁵ being H and R² is the group R⁴, with R⁶ being CH₃;
ix) compound of Formula (I) wherein R¹ is OH and R² is OH;
x) compound of Formula (I) wherein R¹ is CH₃ and R² is CH₃;
xi) compound of Formula (I) wherein R¹ is OCH₃ and R² is OCH₃;
xii) compound of Formula (I) wherein R¹ is OH and R² is the group R⁴, with R⁶ being CH₃;
xiii) compound of Formula (I) wherein R¹ is CH₃ and R² is the group R⁴, with R⁶ being CH₃;
xiv) compound of Formula (I) wherein R¹ is the group R³, with R⁵ being OH and R² is CH₃;
xv) compound of Formula (I) wherein R¹ is the group R³, with R⁵ being any methyl- or ethyl ester and R² is CH₃;
xvi) compound of Formula (I) wherein R¹ is the group R³, with R⁵ being CH₃ and R² being any methyl- or ethyl ester.

5. A compound of Formula (II) according to claim 1 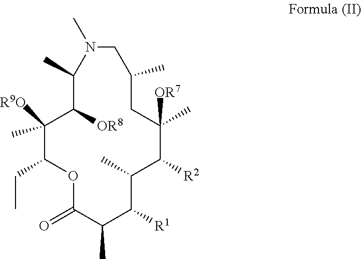
wherein
R¹ is OH, CH₃, OCH₃, a C₂-C₄ straight or branched alkyl group, or the group R³, which is bounded to Formula (I) via a covalent bonding to oxygen, 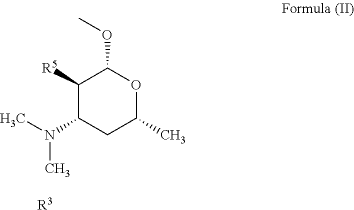
R² is OH, CH₃, OCH₃, a C₂-C₄ straight or branched alkyl group, or the group R⁴, which is bounded to Formula (I) via a covalent bonding to oxygen, 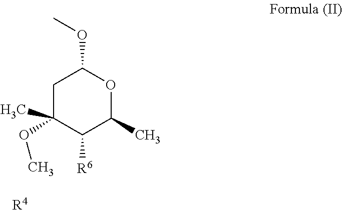
R⁵, R⁶, R⁷, R⁸, and R⁹ has the same meanings as given above,
or a pharmaceutically derivative thereof, tautomers and stereoisomers thereof, or a pharmaceutically acceptable salt thereof,
with the provisio that
R⁵ and R⁶ cannot both be OH.

6. The compound of claim 5, wherein
R¹ is the group R³, where R⁵ is H, OH or CH₃, and
R² is OH, CH₃, or OCH₃.

7. The compound of claim 5, wherein
R¹ is OH, CH₃, OCH₃, and R² is the group R⁴, with R⁶ being H, OH or CH₃.

8. The compound of claim 5, selected from the group consisting of
i) compound of Formula (II) wherein R¹ is the group R³, with R⁵ being CH₃, and R² is the group R⁴, with R⁸ being OH;
ii) compound of Formula (II) wherein R¹ is the group R³, with R⁵ being OH and R² is the group R⁴, with R⁶ being CH₃;
iii) compound of Formula (II) wherein R¹ is the group R³, with R⁵ being CH₃ and R² is the group R⁴, with R⁸ being CH₃;
iv) compound of Formula (II) wherein R¹ is the group R³, with R⁵ being OH and R² is the group R⁴, with R⁶ being H;
v) compound of Formula (II) wherein R¹ is the group R³, with R⁵ being H and R² is the group R⁴, with R⁶ being OH;
vi) compound of Formula (II) wherein R¹ is the group R³, with R⁵ being H and R² is the group R⁴, with R⁶ being H;
vii) compound of Formula (II) wherein R¹ is the group R³, with R⁵ being CH₃ and R² is the group R⁴, with R⁶ being H;
viii) compound of Formula (II) wherein R¹ is the group R³, with R⁵ being H and R² is the group R⁴, with R⁶ being CH₃;
ix) compound of Formula (II) wherein R¹ is OH and R² is OH;
x) compound of Formula (II) wherein R¹ is CH₃ and R² is CH₃;
xi) compound of Formula (II) wherein R¹ is OCH₃ and R² is OCH₃;
xii) compound of Formula (II) wherein R¹ is OH and R² is the group R⁴, with R⁶ being CH₃;
xiii) compound of Formula (II) wherein R¹ is CH₃ and R² is the group R⁴, with R⁶ being CH₃;
xiv) compound of Formula (II) wherein R¹ is the group R³, with R⁵ being OH and R² is CH₃;
xv) compound of Formula (II) wherein R¹ is the group R³, with R⁵ being any methyl- or ethyl ester and R² is CH₃; or
xvi) compound of Formula (II) wherein R¹ is the group R³, with R⁵ being CH₃ and R² being any methyl- or ethyl ester.

9. A pharmaceutical composition comprising a compound as defined in claim 1, and a pharmaceutical acceptable excipient or diluent.

10. The compound according to claim 1 for use as a medicament.

11. The compound according to claim 1, a pharmaceutical composition according to claim 9, or a medicament according to claim 10, for use in treatment of asthma, COPD, diffuse panbronchiolitis, adult respiratory distress syndrome, inflammatory bowel disease, Crohn's disease, chronic bronchitis, and cystic fibrosis.
